SEQUENCE LISTING (59 sequences)

SEQ ID NO: 1 - DNA, 26 bp, synthetic construct
  misc_feature 1..26: note = miRNA target sequence; related miRNA: miR-17-5p
  source 1..26: mol_type = other DNA
agactacctgcactgtaagcactttg

SEQ ID NO: 2 - DNA, 23 bp, synthetic construct
  misc_feature 1..23: note = miRNA target sequence; related miRNA: miR-20a-5p
  source 1..23: mol_type = other DNA
ctacctgcactataagcacttta

SEQ ID NO: 3 - DNA, 22 bp, synthetic construct
  misc_feature 1..22: note = miRNA target sequence; related miRNA: miR-21-5p
  source 1..22: mol_type = other DNA
tcaacatcagtctgataagcta

SEQ ID NO: 4 - DNA, 23 bp, synthetic construct
  misc_feature 1..23: note = miRNA target sequence; related miRNA: miR-221a-3p
  source 1..23: mol_type = other DNA
gaaacccagcagacaatgtagct

SEQ ID NO: 5 - DNA, 23 bp, synthetic construct
  misc_feature 1..23: note = miRNA target sequence; related miRNA: miR-18a-5p
  source 1..23: mol_type = other DNA
ctatctgcactagatgcacctta

SEQ ID NO: 6 - DNA, 23 bp, synthetic construct
  misc_feature 1..23: note = miRNA target sequence; related miRNA: miR-19a-3p
  source 1..23: mol_type = other DNA
tcagttttgcatagatttgcaca

SEQ ID NO: 7 - DNA, 20 bp, synthetic construct
  misc_feature 1..20: note = miRNA target sequence; related miRNA: miR-199-3p
  source 1..20: mol_type = other DNA
ccaatgtgcagactactgta

SEQ ID NO: 8 - DNA, 20 bp, synthetic construct
  misc_feature 1..20: note = miRNA target sequence; related miRNA: miR-33-5p
  source 1..20: mol_type = other DNA
gcaatgcaactacaatgcac

SEQ ID NO: 9 - DNA, 21 bp, synthetic construct
  misc_feature 1..21: note = miRNA target sequence; related miRNA: miR-218b-5p
  source 1..21: mol_type = other DNA
acatggttagatcaagcacaa

SEQ ID NO: 10 - DNA, 23 bp, synthetic construct
  misc_feature 1..23: note = miRNA target sequence; related miRNA: miR-103-3p
  source 1..23: mol_type = other DNA
tcatagccctgtacaatgctgct

SEQ ID NO: 11 - DNA, 22 bp, synthetic construct
  misc_feature 1..22: note = miRNA target sequence; related miRNA: miR-125b-5p
  source 1..22: mol_type = other DNA
tcacaagttagggtctcaggga

SEQ ID NO: 12 - DNA, 24 bp, synthetic construct
  misc_feature 1..24: note = miRNA target sequence; related miRNA: miR-222a
  source 1..24: mol_type = other DNA
gagacccagtagccagatgtagct

SEQ ID NO: 13 - DNA, 22 bp, synthetic construct
  misc_feature 1..22: note = miRNA target sequence; related miRNA: miR-148a-3p
  source 1..22: mol_type = other DNA
acaaagttctgtagtgcactga

SEQ ID NO: 14 - DNA, 23 bp, synthetic construct
  misc_feature 1..23: note = miRNA target sequence; related miRNA: miR-221-3p
  source 1..23: mol_type = other DNA
gaaacccagcagacaatgtagct

SEQ ID NO: 15 - DNA, 22 bp, synthetic construct
  misc_feature 1..22: note = miRNA target sequence; related miRNA: miR-10a-5p
  source 1..22: mol_type = other DNA
acaaattcggatctacagggta

SEQ ID NO: 16 - DNA, 21 bp, synthetic construct
  misc_feature 1..21: note = miRNA target sequence; related miRNA: miR-214
  source 1..21: mol_type = other DNA
ctgcctgtctgtgcctgctgt

SEQ ID NO: 17 - DNA, 23 bp, synthetic construct
  misc_feature 1..23: note = miRNA target sequence; related miRNA: miR-181a-5p
  source 1..23: mol_type = other DNA
actcaccgacagcgttgaatgtt

SEQ ID NO: 18 - DNA, 21 bp, synthetic construct
  misc_feature 1..21: note = miRNA target sequence; related miRNA: miR-92-3p
  source 1..21: mol_type = other DNA
caggccgggacaagtgcaata

SEQ ID NO: 19 - DNA, 21 bp, synthetic construct
  misc_feature 1..21: note = miRNA target sequence; related miRNA: miR-1465
  source 1..21: mol_type = other DNA
aagcacccagcacctctgaaa

SEQ ID NO: 20 - DNA, 22 bp, synthetic construct
  misc_feature 1..22: note = miRNA target sequence; related miRNA: miR-1559-5p
  source 1..22: mol_type = other DNA
ggagtagcatacaagcatcgaa

SEQ ID NO: 21 - DNA, 21 bp, synthetic construct
  misc_feature 1..21: note = miRNA target sequence; related miRNA: miR-1416-5p
  source 1..21: mol_type = other DNA
cacagcggcatgagttaagga

SEQ ID NO: 22 - DNA, 22 bp, synthetic construct
  misc_feature 1..22: note = miRNA target sequence; related miRNA: miR-1451-5p
  source 1..22: mol_type = other DNA
gcggtaacttgctcctgtgcga

SEQ ID NO: 23 - DNA, 22 bp, synthetic construct
  misc_feature 1..22: note = miRNA target sequence; related miRNA: miR-1720-3p
  source 1..22: mol_type = other DNA
tcagaccgacctctcgttgctt

SEQ ID NO: 24 - DNA, 23 bp, synthetic construct
  misc_feature 1..23: note = miRNA target sequence; related miRNA: miR-1596-5p
  source 1..23: mol_type = other DNA
acaaagatggattgaaggagaat

SEQ ID NO: 25 - DNA, 22 bp, synthetic construct
  misc_feature 1..22: note = miRNA target sequence; related miRNA: miR-1677-3p
  source 1..22: mol_type = other DNA
aatcctgctcctactgaagtca

SEQ ID NO: 26 - DNA, 23 bp, synthetic construct
  misc_feature 1..23: note = miRNA target sequence; related miRNA: miR1456-5p
  source 1..23: mol_type = other DNA
gcgcgggccgcctccgtcctttc

SEQ ID NO: 27 - DNA, 21 bp, synthetic construct
  misc_feature 1..21: note = miRNA target sequence; related miRNA: miR-10000
  source 1..21: mol_type = other DNA
gcggcgccacatctgtccgcg

SEQ ID NO: 28 - DNA, 23 bp, synthetic construct
  misc_feature 1..23: note = miRNA target sequence; related miRNA: miR-2131-5p
  source 1..23: mol_type = other DNA
gctgtttccgtgcacttctgcat

SEQ ID NO: 29 - DNA, 22 bp, synthetic construct
  misc_feature 1..22: note = miRNA target sequence; related miRNA: miR-9999
  source 1..22: mol_type = other DNA
aaaatgcggacctcgtccacgc

SEQ ID NO: 30 - DNA, 21 bp, synthetic construct
  misc_feature 1..21: note = miRNA target sequence; related miRNA: miR-1677-5p
  source 1..21: mol_type = other DNA
attgacttcagcggtgcagga

SEQ ID NO: 31 - DNA, 24 bp, synthetic construct
  misc_feature 1..24: note = miRNA target sequence; related miRNA: miR-1464
  source 1..24: mol_type = other DNA
tccgaggcggccctgcaaacagca

SEQ ID NO: 32 - DNA, 23 bp, synthetic construct
  misc_feature 1..23: note = miRNA target sequence; related miRNA: miR-1779-3p
  source 1..23: mol_type = other DNA
ctcaggtgttccagtccacgtct

SEQ ID NO: 33 - DNA, 22 bp, synthetic construct
  misc_feature 1..22: note = miRNA target sequence; related miRNA: miR-1467-3p
  source 1..22: mol_type = other DNA
gatcccagttactctggtgtga

SEQ ID NO: 34 - DNA, 23 bp, synthetic construct
  misc_feature 1..23: note = miRNA target sequence; related miRNA: miR-1451-3p
  source 1..23: mol_type = other DNA
gcctctcacagcagcgagttacg

SEQ ID NO: 35 - DNA, 21 bp, synthetic construct
  misc_feature 1..21: note = miRNA target sequence; related miRNA: miR-1680-3p
  source 1..21: mol_type = other DNA
tcagcctcctgataccgcctg

SEQ ID NO: 36 - DNA, 21 bp, synthetic construct
  misc_feature 1..21: note = miRNA target sequence; related miRNA: miR-1786
  source 1..21: mol_type = other DNA
agtaacacagcagaaaagaat

SEQ ID NO: 37 - DNA, 22 bp, synthetic construct
  misc_feature 1..22: note = miRNA target sequence; related miRNA: miR-1684-3p
  source 1..22: mol_type = other DNA
agatctccatttcctcatactt

SEQ ID NO: 38 - DNA, 20 bp, synthetic construct
  misc_feature 1..20: note = miRNA target sequence; related miRNA: miR-6606-5p
  source 1..20: mol_type = other DNA
tcccgctcctcccgctcctc

SEQ ID NO: 39 - DNA, 22 bp, synthetic construct
  misc_feature 1..22: note = miRNA target sequence; related miRNA: miR-1662
  source 1..22: mol_type = other DNA
atcccaagtatgatgatgtcaa

SEQ ID NO: 40 - DNA, 20 bp, synthetic construct
  misc_feature 1..20: note = miRNA target sequence; related miRNA: miR-1434
  source 1..20: mol_type = other DNA
aattttccatcatcacgcac

SEQ ID NO: 41 - DNA, 21 bp, synthetic construct
  misc_feature 1..21: note = miRNA target sequence; related miRNA: miR-27b-3p
  source 1..21: mol_type = other DNA
gcagaacttagccactgtgaa

SEQ ID NO: 42 - DNA, 25 bp, synthetic construct
  misc_feature 1..25: note = miRNA target sequence; related miRNA: miR-454-3p
  source 1..25: mol_type = other DNA
agaccctataagcaatattgcacta

SEQ ID NO: 43 - DNA, 22 bp, synthetic construct
  misc_feature 1..22: note = miRNA target sequence; related miRNA: miR-460a-5p
  source 1..22: mol_type = other DNA
cacacagtgtgtacaatgcagg

SEQ ID NO: 44 - DNA, 21 bp, synthetic construct
  misc_feature 1..21: note = miRNA target sequence; related miRNA: miR-239b-5p
  source 1..21: mol_type = other DNA
cagtacttttgtgtagtacaa

SEQ ID NO: 45 - DNA, 21 bp, synthetic construct
  misc_feature 1..21: note = miRNA target sequence; related miRNA: miR-196-5p
  source 1..21: mol_type = other DNA
ccaacaacatgaaactaccta

SEQ ID NO: 46 - DNA, 21 bp, synthetic construct
  misc_feature 1..21: note = miRNA target sequence; related miRNA: miR-128-3p
  source 1..21: mol_type = other DNA
aaagagaccggttcactgtga

SEQ ID NO: 47 - DNA, 23 bp, synthetic construct
  misc_feature 1..23: note = miRNA target sequence; related miRNA: miR-126-3p
  source 1..23: mol_type = other DNA
gcgcattattactcacggtacga

SEQ ID NO: 48 - DNA, 107 bp, synthetic construct
  misc_feature 1..107: note = miRblock-1
  source 1..107: mol_type = other DNA
agactacctgcactgtaagcactttgatcgtcatagccctgtacaatgctgcttcgatcacaagttagggtctcagggacgatgagacccagtagccagatgtagct

SEQ ID NO: 49 - DNA, 102 bp, synthetic construct
  misc_feature 1..102: note = miRblock-2
  source 1..102: mol_type = other DNA
ctacctgcactataagcactttaatcgtcaacatcagtctgataagctatcgaacaaagttctgtagtgcactgacgatgaaacccagcagacaatgtagct

SEQ ID NO: 50 - DNA, 105 bp, synthetic construct
  misc_feature 1..105: note = miRblock-scrbl2
  source 1..105: mol_type = other DNA
gaatgtatattccgcctgctcacaatcgattagcttaccatggtcctccagtcgaggcagttgattaactggcaacgcgatactggaggacctatctggcacaag

SEQ ID NO: 51 - DNA, 106 bp, synthetic construct
  misc_feature 1..106: note = miRblock-37
  source 1..106: mol_type = other DNA
ctacctgcactataagcactttaatcgtcaacatcagtctgataagctatcgaagactacctgcactgtaagcactttgcgattcagttttgcatagatttgcaca

SEQ ID NO: 52 - DNA, 106 bp, synthetic construct
  misc_feature 1..106: note = miRblock-38
  source 1..106: mol_type = other DNA
ctatctgcactagatgcaccttaatcgtcaacatcagtctgataagctatcgaagactacctgcactgtaagcactttgcgattcagttttgcatagatttgcaca

SEQ ID NO: 53 - DNA, 102 bp, synthetic construct
  misc_feature 1..102: note = miRblock-39
  source 1..102: mol_type = other DNA
ctatctgcactagatgcaccttaatcgccaatgtgcagactactgtatcgaactacctgcactgtaagcactttgcgattcagttttgcatagatttgcaca

SEQ ID NO: 54 - DNA, 100 bp, synthetic construct
  misc_feature 1..100: note = miRblock-41
  source 1..100: mol_type = other DNA
gcaatgcaactacaatgcacatcgacatggttagatcaagcacaatcgaactacctgcactgtaagcactttgcgatgaaacccagcagacaatgtagct

SEQ ID NO: 55 - DNA, 40 bp, synthetic construct
  misc_feature 1..40: note = PrS promoter
  source 1..40: mol_type = other DNA
aaaaattgaaattttattttttttttttggaatataaata

SEQ ID NO: 56 - DNA, 124 bp, synthetic construct
  misc_feature 1..124: note = Pr13.5long promoter
  source 1..124: mol_type = other DNA
taaaaatagaaactataatcatataatagtgtaggttggtagtattgctcttgtgactagagactttagttaaggtactgtaaaaatagaaactataatcatataatagtgtaggttggtagta

SEQ ID NO: 57 - DNA, 104 bp, synthetic construct
  misc_feature 1..104: note = Pr7.5 promoter
  source 1..104: mol_type = other DNA
tccaaacccacccgctttttatagtaagtttttcacccataaataataaatacaataattaatttctcgtaaaagtagaaaatatattctaatttattgcacgg

SEQ ID NO: 58 - DNA, 227 bp, synthetic construct
  misc_feature 1..227: note = PrLE1 promoter
  source 1..227: mol_type = other DNA
gttttgaaaatttttttataataaatatccggtaaaaattgaaaaactattctaatttattgcacggtccggtaaaaattgaaaaactattctaatttattgcacggtccggtaaaaattgaaaaactattctaatttattgcacggtccggtaaaaattgaaaaactattctaatttattgcacggtccggtaaaaattgaaaaactattctaatttattgcacgg

SEQ ID NO: 59 - DNA, 112 bp, synthetic construct
  misc_feature 1..112: note = PrH5m promoter
  source 1..112: mol_type = other DNA
tacttaaaaattgaaaataaatacaaaggttcttgagggttgtgttaaattgaaagcgagaaataatcataaataatttcattatcgcgatatccgttaagtttgtatcgta

(19) United States
(12) Patent Application Publication
(10) Pub. No.: US 2024/0392257 A1
(43) Pub. Date: Nov. 28, 2024

(54) Utilization of Micro-RNA for Downregulation of Cytotoxic Transgene Expression by Modified Vaccinia Virus Ankara (MVA)

(71) Applicant: Bavarian Nordic A/S, Hellerup (DK)

(72) Inventors: Jürgen Hausmann, Gundelfingen (DE); Markus Kalla, Penzberg (DE); Marc Schweneker, Munich (DE); Matthias Habjan, Puchheim (DE)

(73) Assignee: Bavarian Nordic A/S, Hellerup (DK)

(21) Appl. No.: 18/688,756

(22) PCT Filed: Sep. 2, 2022
(86) PCT No.: PCT/EP2022/074510

§ 371 Date: Mar. 2, 2024

(30) Foreign Application Priority Data
Sep. 3, 2021 (EP) 21194940.9

(51) Int. Cl. C12N 7/00; A61K 39/00; A61K 39/285; C12N 15/113

(52) U.S. Cl. CPC C12N 7/00; A61K 39/285; C12N 15/113; A61K 2039/5256; C12N 2310/141; C12N 2710/24134; C12N 2710/24151; C12N 2760/00022

(57) ABSTRACT

The invention relates to a recombinant Modified Vaccinia Virus Ankara (MVA) comprising a series of miRNA target sequences arranged in a miRblock that is linked to a transgene, wherein each miRNA target sequence corresponds to a miRNA in a eukaryotic MVA producer cell. The present invention also relates to medical uses of the recombinant MVA.

### TECHNICAL FIELD

The present invention relates to the field of viral vectors, particularly to viral vector-based vaccines. More specifically, the present invention relates to a recombinant Modified Vaccinia Virus Ankara (MVA) comprising a series of microRNA (miRNA) target sequences arranged in a so-called miRblock that is linked to a transgene, wherein each miRNA target sequence corresponds to a miRNA expressed in a eukaryotic MVA producer cell. The present invention also relates to medical uses of the recombinant MVA.

### BACKGROUND

A common problem of recombinant viral vectors is the negative effects that transgene products expressed by such vectors can exert on cellular processes in vector producer cells. This can ultimately lead to impaired yields of a given recombinant viral vector (1). Cytotoxic effects can be the results of expression of a single or of multiple transgenes, or even a combination of transgene products showing no cytotoxic effects when expressed separately. The problem of cytotoxic transgene products reducing the viral vector yields also pertains to recombinant MVA vectors, which were derived from the prototype species vaccinia virus of the Orthopoxvirus genus within the family Poxviridae. Impaired replication has for example been observed for an HIV-env expressing MVA (2).

MVA-BN® is a well-characterized virus vector isolated from a Modified Vaccinia Virus Ankara (MVA) virus stock. MVA originates from the dermal vaccinia virus Ankara strain (Chorioallantois vaccinia virus Ankara, CVA) that is a replicating vaccinia virus (3). By serial propagation of CVA over more than 570 passages on primary chicken embryo fibroblasts (CEFs or CEF cells), the attenuated CVA-derived virus MVA was obtained. This MVA was further passaged by Bavarian Nordic resulting in a further attenuated MVA strain, MVA-BN® (4). MVA-BN® lacks approximately 15% of the genome compared to ancestral CVA virus (loss of 31 kb resulting in six major deletion sites). These deletions affect a number of virulence and host range genes, as well as the gene for Type A inclusion bodies. MVA-BN® can attach to, enter and express very efficiently virally encoded genes in human cells. However, assembly and release of progeny virus does not occur in human cells. Therefore, MVA-BN® is an important and versatile vaccine vector able to efficiently express antigen-encoding transgenes for use in vaccination approaches that target diseases with hitherto unmet medical need (for example Ebola virus disease (5)). Preparations of MVA-BN® and derivatives have been administered to many types of animals and to more than 10.500 human subjects in clinical studies, including immunodeficient individuals, without any serious adverse events.

The reduction in viral yields of some MVA recombinants expressing cytotoxic transgenes can vary over a wide range resulting in significant reduction of viral yields up to severe replication impairment or even failure to generate particularly recombinant MVAs expressing certain transgenes. Impairment of recombinant MVA replication can be triggered by just a single, very cytotoxic transgene but also by the combination of multiple transgenes, which might not appear to be cell-toxic individually upon MVA-mediated expression, but the minimal cell-toxic effects of which appear to add up or even synergize to result in significantly decreased MVA yields. The failure to generate recombinant MVAs containing certain transgenes might be attributed to the selective disadvantage conferred by a deleterious transgene to the replication process of an MVA recombinant to an extent that its replication is so inefficient that it cannot be successfully selected and isolated from the parental MVA background. In addition, the genetic stability of the transgenic insert or the genome of the viral vector expressing this transgene can be compromised by the expression of deleterious transgenes during virus vector production (1, 2).

Thus, there is a need for recombinant MVAs capable of expressing multiple, potentially cytotoxic transgenes while the capability of virus replication is impaired as little as possible.

We aimed to evaluate whether the microRNA machinery of the MVA vector producer cells might be utilized to downregulate transgene expression during recombinant MVA production.

MicroRNAs (miRNAs) are small non-coding RNAs typically 21-23 nucleotides (nt) in length that are produced by all eukaryotic cells for post-transcriptional regulation of gene expression. They are encoded in the cellular genome as long non-coding RNAs of some 100 to 1000 nucleotides called primary miRNA (pri-miRNA) and are trimmed in the nucleus by the RNase Drosha to about 80 nt hairpin structures called precursor miRNA (pre-miRNA). These hairpin-RNAs are further trimmed in the cytoplasm by the RNase Dicer to yield the mature 21-23 nt miRNAs. The mature miRNAs are loaded into the so-called RISC multi-protein complex that mediates the miRNA effects. Binding of miRNAs to their cognate target sequences within the coding sequence or the 5′ or the 3′-untranslated region (UTR) of cellular mRNAs leads to either a reduction in their translatability when the match is imperfect or even to mRNA degradation when the match is perfect. In the latter process, the miRNAs mediating mRNA degradation are not degraded themselves and are retrieved by the microRNA effector machinery. Thus, they can initiate a new cycle of mRNA degradation upon recognition of their target sequences. Of note, downmodulation of cellular protein levels by miRNAs is usually lower than two-fold (6, 7), but these effects can be enhanced e.g. by perfect target matching and by tandem arrangement of target sequences (8).

Many miRNA genes are very highly conserved across the animal kingdom while there are also miRNAs that are lineage-, species- or even tissue-specific. There is a number of examples where viruses have been engineered to be targeted by cellular miRNAs with the general goal to achieve a specific tissue tropism or to prevent their replication in non-tumor tissue. Examples of successful replication restriction are poliovirus that was modified by insertion of two separate single miRNA target sequences, and vesicular stomatitis virus (VSV) that was modified by a triple target sequence. These studies indicate that virus replication in vivo can be controlled by miRNAs (9, 10). In a similar way, attenuation of influenza virus or an oncolytic picornavirus was achieved by inserting target sequences of either species-specific or tissue-specific microRNAs into the viral genomes (11, 12). Species-specific attenuation of influenza A virus in mice but not in chicken eggs by miRNA-93 target sequence insertion into an influenza ORF has also been demonstrated (13).

Apart from deliberately attenuating virus replication by miRNA targeting, the microRNA machinery has also been used to silence transgene expression of a viral gene therapy vector. Packaging of adeno-associated virus (AAV) vector genomes into vector particles can severely be impaired by expression of the encoded transgene under the control of a eukaryotic promoter if the transgene product is cytotoxic. miRNA-mediated downregulation of cytotoxic transgene expression increased packaging efficiencies and yields of the respective AAV (14). Hitherto, this approach has been suggested to only be effective by overexpressing artificial miRNAs (amiRNAs) in a pri-miRNA-like scaffold (14) or as short hairpin RNAs (shRNAs) (15) to enhance AAV yields. AAV vectors are typically produced by transfection of a set of plasmids encoding the vector genome and the helper functions, and it was thus technically feasible to co-transfect amiRNA or shRNA expression plasmids. The miRNA transfection approach, however, is not feasible for MVA or generally for poxvirus-based vectors that are propagated by infection of susceptible producer cells. In the case of the CEF-adapted MVA, common producer cells are primary CEF cells or a few avian cell lines like the continuous chicken fibroblast cell line DF-1. Primary CEFs show very limited transfection efficiency and thus the transfection procedure in general is not ideal in an industrial-scale production process.

Therefore, we aimed to make use of cell-endogenous miRNAs in primary CEFs to downregulate transgene expression driven by the MVA vector during vector production to achieve higher recombinant MVA yields for vaccination purposes.

There is only limited publicly available knowledge regarding the mutual interaction of the miRNA machinery and vaccinia virus infection. It appears that vaccinia virus downregulates the cellular miRNA machinery on different levels including induction of miRNA degradation (16, 17) as well as downregulation of the cellular RNase Dicer (17, 18) required for miRNA biogenesis. This suggested that miRNAs would not be suitable to modulate vaccinia virus-driven transgene expression. There is one example of targeted miRNA-mediated downregulation of expression of a vaccinia virus protein named B5 with the goal to impair B5-dependent vaccinia virus morphogenesis and consequently progeny virus production (19, 20). However, the early/late poxviral B5 promoter driving expression of the B5R gene is not a very strong promoter (21, 22). In contrast, the problem in controlling poxvirus-driven transgene expression in producer cells is magnified by the fact that poxviral promoters used to drive expression of transgenes have been deliberately designed and selected to be as strong as possible to direct the synthesis of maximal amounts of recombinant protein serving as immunogen (for example the widely used synthetic early/late PrS promoter (23) and the strong immediate-early promoter Pr13.5long (24)).

### SUMMARY OF INVENTION

It is an object of the present invention to provide means and methods for producing recombinant MVA at increased yields.

The problem underlying the invention is solved by the provision of a recombinant MVA modified such that the expression of transgenes is downregulated during MVA propagation in susceptible producer cells. In particular, the invention is defined by the appended claims and by the following aspects and their embodiments.

In a first aspect, the invention provides a recombinant Modified Vaccinia Virus Ankara (MVA) comprising a nucleotide sequence comprising a transgene operably linked to a poxvirus promoter, the nucleotide sequence further comprising a miRNA target sequence that is linked to the transgene, wherein the miRNA target sequence corresponds to a miRNA in a eukaryotic MVA producer cell.

In a second aspect, the invention provides a recombinant MVA comprising a nucleotide sequence comprising a transgene operably linked to a poxvirus promoter, the nucleotide sequence further comprising a series of miRNA target sequences arranged in a miRblock that is linked to the transgene, wherein each miRNA target sequence in the miRblock corresponds to a miRNA in a eukaryotic MVA producer cell.

In further aspect, the invention provides a recombinant MVA comprising a transcriptional unit comprising a nucleotide sequence comprising a transgene operably linked to a poxvirus promoter, the nucleotide sequence further comprising a miRNA target sequence that is linked to the transgene, or a series of miRNA target sequences arranged in a miRblock that is linked to the transgene, wherein the miRNA target sequence or each miRNA target sequence in the miRblock corresponds to a miRNA in a eukaryotic MVA producer cell.

In yet a further aspect, the invention provides a recombinant MVA comprising a first and a second transcriptional unit, or more transcriptional units, each transcriptional unit comprising a nucleotide sequence comprising a transgene operably linked to a poxvirus promoter, the nucleotide sequence further comprising a miRNA target sequence that is linked to the transgene, or a series of miRNA target sequences arranged in a miRblock that is linked to the transgene, wherein the miRNA target sequence or each miRNA target sequence in the miRblock corresponds to a miRNA in a eukaryotic MVA producer cell.

In another aspect, the invention provides a transcriptional unit, preferably suitable for use in a recombinant MVA, comprising a nucleotide sequence comprising a transgene operably linked to a poxvirus promoter, the nucleotide sequence further comprising a miRNA target sequence that is linked to the transgene, or a series of miRNA target sequences arranged in a miRblock that is linked to the transgene, wherein the miRNA target sequence or each miRNA target sequence in the miRblock corresponds to a miRNA in a eukaryotic MVA producer cell.

In yet another aspect, the invention provides of a series of miRNA target sequences arranged in a miRblock, preferably suitable for use in a recombinant MVA, wherein each miRNA target sequence corresponds to a miRNA in a eukaryotic MVA producer cell.

In yet another aspect, the invention provides a plasmid comprising a nucleotide sequence comprising a transgene operably linked to a promoter which is active in a eukaryotic producer cell, the nucleotide sequence further comprising a miRNA target sequence that is linked to the transgene, or a series of miRNA target sequences arranged in a miRblock that is linked to the transgene, wherein the miRNA target sequence or each miRNA target sequence in the miRblock corresponds to a miRNA in the eukaryotic producer cell.

In yet another aspect, the invention provides a process for producing a recombinant MVA according to the invention, comprising the steps of:
(1) providing a miRNA target sequence or a series of miRNA target sequences arranged in a miRblock according to the invention;
(2) preparing a transcriptional unit according to the invention using the miRNA target sequence or the miRblock provided in step (1);
(3) inserting the transcriptional unit prepared in step (2) into an MVA;
(4) infecting a eukaryotic MVA producer cell with the MVA obtained in step (3) and propagating the same;
(5) harvesting the recombinant MVA propagated in step (4).

In yet another aspect, the invention provides a recombinant MVA produced by a process according to the invention.

In yet another aspect, the invention provides a use of a recombinant MVA according to the invention for industrial-scale production of a vaccine.

In yet another aspect, the invention provides a use of a miRNA target sequence according to the invention for downregulation of the expression of an MVA encoded transgene in a eukaryotic MVA producer cell, particularly for industrial-scale production of a vaccine.

In yet another aspect, the invention provides a use of a series of miRNA target sequences arranged in a miRblock according to the invention for downregulation of the expression of an MVA encoded transgene in a eukaryotic MVA producer cell, particularly for large-scale production of a vaccine.

In yet another aspect, the invention provides a pharmaceutical composition or a vaccine comprising the recombinant MVA according to the invention, optionally further comprising a pharmaceutically acceptable carrier or excipient.

In yet another aspect, the invention provides a recombinant MVA according to the invention for use as a medicament or a vaccine, preferably for use in the treatment or prevention of a disease.

In yet another aspect, the invention provides a recombinant MVA according to the invention for use in the treatment or prevention of an infectious disease or cancer.

In yet another aspect, the invention provides a use of a recombinant MVA according to the invention for the manufacture of a medicament or vaccine for use in the treatment or prevention of an infectious disease or cancer.

In yet another aspect, the invention provides a method of treating or preventing an infectious disease or cancer in a subject, the method comprising administering to the subject a recombinant MVA according to the invention.

These aspects and their embodiments will be described in further detail in connection with the description of invention.

### BRIEF DESCRIPTION OF DRAWINGS/FIGURES

FIG. 1 illustrates the design of plasmid inserts expressing EGFP and containing miRNA target sequences arranged in hetero-oligomeric miRblocks in the EGFP 3′-UTR.

EGFP plasmids without miRNA target sequences (“EGFP no miRb”) (A), with hetero-oligomeric miRblock-1 (B) and -2 (C), and with a control miRblock (“EGFP-scrbl2”) containing four scrambled miRNA target sequences (D). pCMV=human cytomegalovirus immediate-early promoter/enhancer; EGFP=enhanced green fluorescent protein: SV40 polyA=polyadenylation signal from simian virus-40; nt=nucleotides; ORF=open reading frame.

FIG. 2 shows a comparison of miRblock effects on plasmid-driven EGFP expression in primary CEF cells.

CEF cells in VP-SFM medium were seeded on day 0 in 96-well plates (4×10⁴ cells/well) at 37° C. Cells were co-transfected on day 1 in triplicates with EGFP- and blue fluorescence protein (BFP)-encoding plasmids. EGFP-encoding plasmids with 10 different hetero-oligomeric miRblocks in the 3′-UTR of the EGFP gene are named miRb-1 to miRb-10 (for miRNA target sequences see Table 4). Transfection with a plasmid encoding EGFP containing no miRblock served as a reference for EGFP expression (“no miRb”). Cells were analyzed for EGFP and BFP expression by flow cytometry on day 2. Geometric mean fluorescence intensities (GMFI) of EGFP (top) and BFP (bottom) of BFP-positive cells are shown (geometric mean (GM) with error bars indicating geometric standard deviation (geoSD)). Percentages indicate % EGFP expression level relative to that of the EGFP expression reference not containing a miRblock.

FIG. 3 shows an analysis of miRblock effects at 30° C. and 37° C. on EGFP expression after plasmid transfection in CEF and DF-1 cells.

CEF cells (left) in VP-SFM medium and DF-1 cells (right) in DMEM/10% FCS were seeded on day 0 in 96-well plates (4×10⁴ cells/well). Cells were co-transfected on day 1 in triplicates with plasmids encoding EGFP (“miRb-1”, “miRb-2”, miRblock control “scrbl”) and BFP. Transfection with a plasmid encoding EGFP containing no miRblock served as EGFP expression reference (“no miRb”). Cells were incubated at 30° C. or 37° C. and analyzed for EGFP and BFP expression by flow cytometry 23 hours after transfection. GMFI of EGFP (top) and BFP (bottom) of BFP-positive cells are shown (GM with geoSD). % EGFP expression levels relative to the EGFP expression reference.

FIG. 4 illustrates the design of recombinant MVA inserts encoding EGFP and containing miRNA target sequences arranged in hetero-oligomeric miRblocks in the EGFP 3′-UTR.

Recombinant MVA encoding EGFP without miRNA target sequences (“EGFP”), with hetero-oligomeric miRblock-1 and -2 (“EGFP-miRb-1”, “EGFP-miRb-2”), and with a control miRblock (“EGFP-scrbl2”). PrS=synthetic poxviral early/late promoter; RFP=red fluorescence protein; gpt=guanine phosphoribosyl transferase; nt=nucleotides; TTS=early transcription termination signal; IGR=intergenic region in the MVA genome.

FIG. 5 shows an analysis of miRblock effects on EGFP expression levels by recombinant MVA.

CEF cells (left) in VP-SFM medium and DF-1 cells (right) in DMEM/10% FCS were seeded on day 0 in 96-well plates (4×10⁴ cells/well). On day 1, cells were infected in triplicates with EGFP and RFP expressing MVA-BN® recombinants containing no miRblock (“no miRb”, EGFP expression reference), miRblock-1 or -2 (“miRb-1”, “miRb-2”), or miRblock-scrbl2 control (“scrbl2”) at a multiplicity of infection (MOI) of 5. Cells were either incubated at 30° C. or 37° C. during infection. At 19 hours p.i., cells were trypsinized, washed, and resuspended in PBS-FACS-FORM (1% FCS, 0.1% NaN₃, 1% paraformaldehyde (PFA)). Expression of EGFP and RFP was analyzed by flow cytometry. GMFI of EGFP (top) and RFP (bottom) of RFP-positive cells are shown (GM with geoSD). % EGFP expression levels relative to the EGFP expression reference.

FIG. 6 shows an analysis of miRblock effects on EGFP expression after infection with recombinant MVA at different MOIs.

CEF cells (left) in VP-SFM medium (left) and DF-1 cells (right) in DMEM/10% FCS (right) were seeded in triplicates on day 0 in 96-well plates (4×10⁴ cells/well). On day 1, cells were infected with EGFP and RFP expressing MVA-BN recombinants containing no miRblock (“no miRb”, EGFP expression reference), miRblock-1 or -2 (“miRb-1”, “miRb-2”), or miRblock-scrbl2 control (“scrbl2”) at a MOI of 5, 1, or 0.2 as indicated. At 6 hours post infection, cells were trypsinized, washed, and resuspended in PBS-FACS-FORM (1% FCS, 0.1% NaN₃, 1% PFA). Expression of EGFP was analyzed by flow cytometry. GMFI of EGFP (top; GM with geoSD) and % GMFI EGFP expression levels relative to the EGFP expression reference (bottom; mean with standard error of the mean (SEM)) are shown. Expression of RFP was not recorded.

FIG. 7 shows an analysis of miRblock effects on EGFP expression over time (multi-cycle replication) after infection with recombinant MVA at low MOI.

CEF cells in VP-SFM medium were seeded in triplicates on day 0 in 96-well plates (4×10⁴ cells/well). On day 1, cells were infected with EGFP and RFP expressing MVA-BN recombinants containing no miRblock (“no miRb”), miRblock-1 or -2 (“miRb-1”, “miRb-2”), or miRblock-scrbl2 (“scrbl2”, control) at a MOI of 0.1. At the indicated times p.i., cells were trypsinized, washed, and resuspended in PBS-FACS-FORM (1% FCS, 0.1% NaN₃, 1% PFA). Expression of EGFP and RFP was analyzed by flow cytometry. SGMFI of EGFP (top, left) and RFP (top, right) of RFP-positive cells (GM with geoSD) are shown as well as % GMFI EGFP and % GMFI RFP relative to the EGFP expression reference (bottom; mean with SEM).

FIG. 8 illustrates the design of recombinant MVA inserts containing miRNA target sequences arranged in hetero-oligomeric miRblocks and expressing EGFP under different promoters. PrS=synthetic poxviral early/late promoter; Pr13.5long=immediate-early promoter.

FIG. 9 shows a comparison of miRblock effects on EGFP expression under different poxviral promoters.

CEF cells (left) in VP-SFM medium and DF-1 cells (right) in DMEM/10% FCS were seeded on day 0 in 96-well plates (4×10⁴ or 3×10⁴ cells/well). On day 1, cells were infected (MOI 10) in triplicates with EGFP and RFP expressing MVA-BN® recombinants containing miRblock-2 (“miRb-2”) or miRblock-scrbl2 (“scrbl2”, control) with the miRNA targeted EGFP gene either under control of the PrS promoter or the Pr13.5long promoter. At 6 hours and 18 hours p.i., cells were trypsinized, washed, and resuspended in PBS-FACS-FORM (1% FCS, 0.1% NaN₃, 1% PFA). Expression of EGFP and RFP was analyzed by flow cytometry. % GMFI of EGFP relative to EGFP expression levels in RFP-positive cells infected with the “scrbl2” control are shown (mean with SEM). PrS or Pr13.5long promoter and infection time as indicated. % EGFP expression levels relative to the “scrbl2” control.

FIG. 10 shows a comparison of hetero- and related homo-oligomeric miRblocks regarding their effects on plasmid-driven EGFP expression.

CEF cells in VP-SFM medium (4×10⁴ cells/well) were seeded on day 0 in 96-well plates. Cells were co-transfected on day 1 in triplicates with EGFP- and BFP-encoding plasmids. EGFP expression by plasmids containing hetero-oligomeric miRblock-1 or -2 (“miRb-1”, “miRb-2”) or homo-oligomeric miRblock-13 to -20 was analyzed. miRblock-13 to -16 contained triplicate repeats of miRNA target sequences contained in miRblock-1; miRblock-17 to -20 contained quadruple repeats of miRNA target sequences contained in miRblock-2. A plasmid containing no miRblock (“no miRb”) served as EGFP expression reference, a plasmid containing miRblock-scrbl2 (“scrbl2”) served as control. GMFI of EGFP (top) and BFP (bottom) of BFP-positive cells (GM with geoSD) determined by flow cytometry on day 1 after transfection are shown. % EGFP expression relative to the EGFP expression reference. Sets of miRblock-containing plasmids were analyzed in two separate experiments.

FIG. 11 illustrates the design of recombinant MVA inserts expressing EGFP and containing selected miRblock-2 derived miRNA target sequences arranged in homo-oligomeric miRblocks.

Recombinant MVA without miRNA target sequences (“EGFP”), with hetero-oligomeric miRblock-2 (“EGFP-miRb-2”), with homo-oligomeric miRblock-17 or -18 (“EGFP-miRb-17”, “EGFP-miRb-18”), and with a control miRblock (“EGFP-scrbl2”).

FIG. 12 shows a comparison of hetero- and related homo-oligomeric miRblocks regarding their effects on EGFP expression by recombinant MVA.

CEF cells in VP-SFM were seeded on day 0 in 96-well plates (4×10⁴ cells/well). On day 1, cells were infected with MVA-BN® recombinants (MOI 5) containing hetero-oligomeric miRblock-2 (“miRb-2”) or homo-oligomeric miRblock-17 or -18 (“miRb-17”, “miRb-18”). miRblock-17 and -18 contained quadruple repeats of miRNA target sequences contained in miRblock-2. At 18 hours p.i., cells were trypsinized, washed, and resuspended in PBS-FACS-FORM (1% FCS, 0.1% NaN3, 1% PFA). Expression of EGFP and RFP was analyzed by flow cytometry. GMFI of EGFP (left) and RFP (right) expression of RFP-positive cells are shown (GM with geoSD). % EGFP expression levels relative to EGFP expression reference (“no miRb).

FIG. 13 shows a comparison of plasmid-driven EGFP expression controlled by homo-oligomeric miRblocks designed on the basis of selected miRNAs.

CEF cells (left) in VP-SFM medium (4×10⁴ cells/well) and DF-1 cells (right) (3×10⁴ cells/well) in DMEM/10% FCS were seeded on day 0 in 96-well plates. Cells were transfected on day 1 in triplicates with EGFP- and BFP-expressing plasmids. EGFP expression by plasmids containing homo-oligomeric miRblock-25 to -36 composed of quadruple repeats of miRNA target sequences were analyzed. Plasmids containing hetero-oligomeric miRblock-1 or -2 were included for comparison. A plasmid containing no miRblock (“no miRb”) served as EGFP expression reference, a plasmid containing miRblock-scrbl2 (“scrbl2”) served as control. GMFI of EGFP (top) and BFP (bottom) of BFP-positive cells (GM with geoSD) determined by flow cytometry on day 1 after transfection are shown. % EGFP expression relative to EGFP expression reference (“no miRb”). The datasets comprising data for miRblock-25 and -26 are from independent experiments.

FIG. 14 shows an analysis of EGFP downregulation in cells infected with recombinant MVA containing hetero-oligomeric miRblocks composed of most effective miRNA target sequences.

CEF cells (left) (4×10⁴ cells/well) in VP-SFM medium and DF-1 cells (right) (3×10⁴ cells/well) in DMEM/10% FCS were seeded on day 0 in 96-well plates. Cells were transfected on day 1 in triplicates with EGFP and BFP expressing plasmids containing hetero-oligomeric miRblocks. miRblock-37 to -47 were composed of miRNA target sequences from homo-oligomeric miRblock-13, -17-, -18-20 (cf. FIG. 10) and miRblock-25, -26, -31 to -33 (cf. FIG. 13). A plasmid containing hetero-oligomeric miRblock-2 was included for comparison. A plasmid containing no miRblock (“no miRb”) served as EGFP expression reference, a plasmid containing miRblock-scrbl2 (“scrbl2”) served as control. At 24 hours post transfection, cells were analyzed for expression of EGFP and BFP by flow cytometry. GMFI of EGFP (top) and BFP (bottom) of BFP-positive cells (GM with geoSD) are shown. % EGFP expression relative to EGFP expression reference (“no miRb”).

FIG. 15 illustrates the design of recombinant MVA-BN-RSV and modified versions thereof containing miRblocks in the 3′-UTR of RSV-derived transgenes

MVA-BN® recombinants (“MVA-BN-RSV”, “MVA-BN-RSV-miRb1/2”, “MVA-BN-RSV-miRb39/41”) encoding RSV-derived transgenes under the control of different promoters are depicted as indicated. In MVA-BN-RSV, the encoded RSV-derived transgenes are not linked to miRblocks. In MVA-BN-RSV-miRb1/2 and MVA-BN-RSV-miRb39/41, three or all four transgenes are linked to hetero-oligomeric miRblocks, respectively. G(A), G(B)=ORFs for the A and B serotypes of RSV G protein; N=RSV nucleoprotein ORF; 2A=picornavirus-derived self-cleaving 2A peptide; M2-1=M2-1 ORF from the RSV M2 gene; F (A-long BN)=ORF for modified A-long serotype of the RSV-F protein; poxviral promoters as indicated.

FIG. 16 shows an analysis of miRNA-mediated downregulation of MVA encoded RSV G, F, and N/M2-1 transgenes.

1×10⁶ CEF cells in VP-SFM were seeded on day 0. The following day, cells were mock infected or infected with MVA-BN® or recombinants MVA-BN-RSV, MVA-BN-RSV-miRb1/2, or MVA-BN-RSV-miRb39/41 at a MOI of 1. Cell lysates (“CL”) were prepared 12 hours (left) and 18 hours p.i. (right), proteins were separated according to size by SDS-PAGE and analyzed by immunoblotting using an anti-RSV G, anti-RSV F or anti-RSV N antibody, or an anti-D8 VACV antibody (MVA vector control).

FIG. 17 shows the capability of replication of MVA-BN-RSV-miRb1/2 and MVA-BN-RSV-miRb39/41 in comparison with MVA-BN-RSV.

CEF cells were seeded in VP-SFM medium in 6-well plates one day before infection. Confluent monolayers were infected in triplicates at a MOI of 0.1 (left) or 0.01 (right) with MVA recombinants in 1 ml VP-SFM: MVA-BN-RSV, MVA-BN-RSV-miRb1/2 or MVA-BN-RSV-miRb39/41. MVA-BN® wildtype (i.e., non-recombinant) was included for comparison. Infected cells were cultured at 30° C. and were directly frozen at day 3 and day 4 p.i. for subsequent TCID₅₀ titration. Viral yields are indicated as geometric mean of TCID₅₀/2 ml with SD. In the table, fold differences in viral titer calculated from data shown in the figure (top) are indicated.

FIG. 18 shows an analysis of RSV-specific CD8+ T cell responses in peripheral blood cells of mice analyzed by Dextramer staining.

Groups of 10 female BALB/c mice were immunized intramuscularly on day 0 and day 21 with 1×10⁸ TCID₅₀ per mouse with MVA-BN-RSV, MVA-BN-RSV-miRb1/2 or MVA-BN-RSV-miRb39/41. TBS-treated mice (n=5) were included as control. PBMCs were collected on day 7 post prime, day 28 (=day 7 post boost), and day 34 (=day 13 post boost) and stained with MHC class I dextramers specific for the immunodominant epitopes in RSV M2-1 and in the MVA E3 protein (vector control), as well as for expression of the surface markers CD4, CD8, and CD44. Percentages of live CD8+ T cells that are activated (CD44+) and specific for RSV M2-1 (left) or MVA-derived E3 (right) are shown (mean with SEM).

FIG. 19 shows an analysis of RSV-specific T cell responses in splenocytes of mice analyzed by intracellular cytokine staining (ICCS) and ELISpot.

Female BALB/c mice were treated as described for FIG. 18. On day 13 post boost, single cell splenocyte suspensions were prepared for ICCS of T cells and for ELISpot analysis. (A) For ICCS, splenocytes were restimulated for 6 hours with immunodominant peptides from the indicated RSV proteins or the immunodominant MVA epitope derived from MVA E3 (vector control). Frequencies of CD44⁺ IFN-γ⁺ cells of CD8+ T cells after peptide stimulation are shown (mean with SEM). (B) For ELISpot analyses, splenocytes were restimulated with immunodominant RSV G, F, and M2-1-derived peptides or the immunodominant MVA-E3 peptide as indicated. IFN-γ⁺ spot-forming colonies (SFC) per 1×10⁶ splenocytes are shown (mean with SEM).

FIG. 20 shows an analysis of RSV-specific IgG antibody titers and RSV plaque reduction neutralization titers in blood serum from mice.

Female BALB/c mice were treated as described for FIG. 18. Serum was collected one day before immunization (day −1), on day 20 post prime, and day 34 (=day 13 post boost). (A) RSV- and MVA-specific IgG antibodies (left and right, respectively) were detected by ELISA and results are shown as geometric mean titer (GMT) with geoSD. (B) RSV A2 strain specific neutralizing antibody titers in serum samples were determined by plaque reduction neutralization test (PRNT) (GMT with geoSD). Percent of seroconversion, defined as appearance of plaque reduction neutralization titer ≥15 for initially seronegative mice, is indicated in the table.

### BRIEF DESCRIPTION OF SEQUENCES

Table 1: miRNA Target Sequences According to SEQ ID NO: 1 to 9.

TABLE 1
miRNA target sequences according to
SEQ ID NO: 1 to 9.

| | miRNA target sequence | |
|---|---|---|
| SEQ | (complementary to | Related |
| ID NO | related miRNA sequence) | miRNA |
| SEQ ID | AGACTACCTGCACTGTAAGCACTTTG | miR-17-5p |
| NO: 1 | | |
| SEQ ID | CTACCTGCACTATAAGCACTTTA | miR-20a-5p |
| NO: 2 | | |
| SEQ ID | TCAACATCAGTCTGATAAGCTA | miR-21-5p |
| NO: 3 | | |
| SEQ ID | GAAACCCAGCAGACAATGTAGCT | miR-221a-3p |
| NO: 4 | | |
| SEQ ID | CTATCTGCACTAGATGCACCTTA | miR-18a-5p |
| NO: 5 | | |
| SEQ ID | TCAGTTTTGCATAGATTTGCACA | miR-19a-3p |
| NO: 6 | | |
| SEQ ID | CCAATGTGCAGACTACTGTA | miR-199-3p |
| NO: 7 | | |
| SEQ ID | GCAATGCAACTACAATGCAC | miR-33-5p |
| NO: 8 | | |
| SEQ ID | ACATGGTTAGATCAAGCACAA | miR-218b-5p |
| NO: 9 | | |

Table 2: miRNA Target Sequences According to SEQ ID NO: 10 to 47.

TABLE 2
miRNA target sequences according to
SEQ ID NO: 10 to 47.

| | miRNA target sequence | |
|---|---|---|
| SEQ | (complementary to | Related |
| ID NO | related miRNA sequence) | miRNA |
| SEQ ID | TCATAGCCCTGTACAATGCTGCT | miR-103-3p |
| NO: 10 | | |
| SEQ ID | TCACAAGTTAGGGTCTCAGGGA | miR-125b-5p |
| NO: 11 | | |
| SEQ ID | GAGACCCAGTAGCCAGATGTAGCT | miR-222a |
| NO: 12 | | |
| SEQ ID | ACAAAGTTCTGTAGTGCACTGA | miR-148a-3p |
| NO: 13 | | |
| SEQ ID | GAAACCCAGCAGACAATGTAGCT | miR-221-3p |
| NO: 14 | | |
| SEQ ID | ACAAATTCGGATCTACAGGGTA | miR-10a-5p |
| NO: 15 | | |
| SEQ ID | CTGCCTGTCTGTGCCTGCTGT | miR-214 |
| NO: 16 | | |
| SEQ ID | ACTCACCGACAGCGTTGAATGTT | miR-181a-5p |
| NO: 17 | | |
| SEQ ID | CAGGCCGGGACAAGTGCAATA | miR-92-3p |
| NO: 18 | | |
| SEQ ID | AAGCACCCAGCACCTCTGAAA | miR-1465 |
| NO: 19 | | |
| SEQ ID | GGAGTAGCATACAAGCATCGAA | miR-1559-5p |
| NO: 20 | | |
| SEQ ID | CACAGCGGCATGAGTTAAGGA | miR-1416-5p |
| NO: 21 | | |
| SEQ ID | GCGGTAACTTGCTCCTGTGCGA | miR-1451-5p |
| NO: 22 | | |
| SEQ ID | TCAGACCGACCTCTCGTTGCTT | miR-1720-3p |
| NO: 23 | | |
| SEQ ID | ACAAAGATGGATTGAAGGAGAAT | miR-1596-5p |
| NO: 24 | | |
| SEQ ID | AATCCTGCTCCTACTGAAGTCA | miR-1677-3p |
| NO: 25 | | |
| SEQ ID | GCGCGGGCCGCCTCCGTCCTTTC | miR1456-5p |
| NO: 26 | | |
| SEQ ID | GCGGCGCCACATCTGTCCGCG | miR-10000 |
| NO: 27 | | |
| SEQ ID | GCTGTTTCCGTGCACTTCTGCAT | miR-2131-5p |
| NO: 28 | | |
| SEQ ID | AAAATGCGGACCTCGTCCACGC | miR-9999 |
| NO: 29 | | |
| SEQ ID | ATTGACTTCAGCGGTGCAGGA | miR-1677-5p |
| NO: 30 | | |
| SEQ ID | TCCGAGGCGGCCCTGCAAACAGCA | miR-1464 |
| NO: 31 | | |
| SEQ ID | CTCAGGTGTTCCAGTCCACGTCT | miR-1779-3p |
| NO: 32 | | |
| SEQ ID | GATCCCAGTTACTCTGGTGTGA | miR-1467-3p |
| NO: 33 | | |
| SEQ ID | GCCTCTCACAGCAGCGAGTTACG | miR-1451-3p |
| NO: 34 | | |
| SEQ ID | TCAGCCTCCTGATACCGCCTG | miR-1680-3p |
| NO: 35 | | |
| SEQ ID | AGTAACACAGCAGAAAAGAAT | miR-1786 |
| NO: 36 | | |
| SEQ ID | AGATCTCCATTTCCTCATACTT | miR-1684-3p |
| NO: 37 | | |
| SEQ ID | TCCCGCTCCTCCCGCTCCTC | miR-6606-5p |
| NO: 38 | | |
| SEQ ID | ATCCCAAGTATGATGATGTCAA | miR-1662 |
| NO: 39 | | |
| SEQ ID | AATTTTCCATCATCACGCAC | miR-1434 |
| NO: 40 | | |
| SEQ ID | GCAGAACTTAGCCACTGTGAA | miR-27b-3p |
| NO: 41 | | |
| SEQ ID | AGACCCTATAAGCAATATTGCACTA | miR-454-3p |
| NO: 42 | | |
| SEQ ID | CACACAGTGTGTACAATGCAGG | miR-460a-5p |
| NO: 43 | | |
| SEQ ID | CAGTACTTTTGTGTAGTACAA | miR-239b-5p |
| NO: 44 | | |
| SEQ ID | CCAACAACATGAAACTACCTA | miR-196-5p |
| NO: 45 | | |
| SEQ ID | AAAGAGACCGGTTCACTGTGA | miR-128-3p |
| NO: 46 | | |
| SEQ ID | GCGCATTATTACTCACGGTACGA | miR-126-3p |
| NO: 47 | | |

Table 3: miRblocks According to SEQ ID NO: 48 to 54 (Underlined: Target Sequence; not Underlined: 4-Nt Linker).

TABLE 3
miRblocks according to SEQ ID NO: 48 to 54

| SEQ | | |
|---|---|---|
| ID NO | miRblock sequence | miRblock |
| SEQ ID | 5′-AGACTACCTGCACTGTAAGCACTTTGAT | miRblock- |
| NO: 48 | CGTCATAGCCCTGTACAATGCTGCTTCGATC | 1 |
| | ACAAGTTAGGGTCTCAGGGACGATGAGACCC | |
| | AGTAGCCAGATGTAGCT-3′ | |
| SEQ ID | 5′-CTACCTGCACTATAAGCACTTTAATCGT | miRblock- |
| NO: 49 | CAACATCAGTCTGATAAGCTATCGAACAAAG | 2 |
| | TTCTGTAGTGCACTGACGATGAAACCCAGCA | |
| | GACAATGTAGCT-3′ | |
| SEQ ID | 5′-GAATGTATATTCCGCCTGCTCACAATCG | miRblock- |
| NO: 50 | ATTAGCTTACCATGGTCCTCCAGTCGAGGCA | scrbl2 |
| | GTTGATTAACTGGCAACGCGATACTGGAGGA | |
| | CCTATCTGGCACAAG-3′ | |
| SEQ ID | 5′-CTACCTGCACTATAAGCACTTTAATCGT | miRblock- |
| NO: 51 | CAACATCAGTCTGATAAGCTATCGAAGACTA | 37 |
| | CCTGCACTGTAAGCACTTTGCGATTCAGTTT | |
| | TGCATAGATTTGCACA-3′ | |
| SEQ ID | 5′-CTATCTGCACTAGATGCACCTTAATCGT | miRblock- |
| NO: 52 | CAACATCAGTCTGATAAGCTATCGAAGACTA | 38 |
| | CCTGCACTGTAAGCACTTTGCGATTCAGTTT | |
| | TGCATAGATTTGCACA-3′ | |
| SEQ ID | 5′-CTATCTGCACTAGATGCACCTTAATCGC | miRblock- |
| NO: 53 | CAATGTGCAGACTACTGTATCGAACTACCTG | 39 |
| | CACTGTAAGCACTTTGCGATTCAGTTTTGCA | |
| | TAGATTTGCACA-3′ | |
| SEQ ID | 5′-GCAATGCAACTACAATGCACATCGACAT | miRblock- |
| NO: 54 | GGTTAGATCAAGCACAATCGAACTACCTGCA | 41 |
| | CTGTAAGCACTTTGCGATGAAACCCAGCAGA | |
| | CAATGTAGCT-3′ | |

(underlined: target sequence; not underlined: 4-nt linker).

### Table 4: Sequences of Poxviral Promoters.

TABLE 4
Sequences of poxviral promoters.

| SEQ | | |
|---|---|---|
| ID NO | Promoter sequence | Promoter |
| SEQ ID | AAAAATTGAA ATTTTATTTT TTTTTTTTGG | PrS |
| NO: 55 | AATATAAATA | |
| SEQ ID | TAAAAATAGAAACTATAATCATATAATAGTGTA | Pr13.5long |
| NO: 56 | GGTTGGTAGTATTGCTCTTGTGACTAGAGACT | |
| | TTAGTTAAGGTACTGTAAAAATAGAAACTATAA | |
| | TCATATAATAGTGTAGGTTGGTAGTA | |
| SEQ ID | TCCAAACCCA CCCGCTTTTT ATAGTAAGTT | Pr7.5 |
| NO: 57 | TTTCACCCAT AAATAATAAA TACAATAATT | |
| | AATTTCTCGT AAAAGTAGAA AATATATTCT | |
| | AATTTATTGC ACGG | |
| SEQ ID | GTTTTGAAAA TTTTTTTATA ATAAATATCC | PrLE1 |
| NO: 58 | GGTAAAAATT GAAAAACTAT TCTAATTTAT | |
| | TGCACGGTCC GGTAAAAATT GAAAAACTAT | |
| | TCTAATTTAT TGCACGGTCC GGTAAAAATT | |
| | GAAAAACTAT TCTAATTTAT TGCACGGTCC | |
| | GGTAAAAATT GAAAAACTAT TCTAATTTAT | |
| | TGCACGGTCC GGTAAAAATT GAAAAACTAT | |
| | TCTAATTTAT TGCACGG | |
| SEQ ID | TACTTAAAAA TTGAAAATAA ATACAAAGGT | PrH5m |
| NO: 59 | TCTTGAGGGT TGTGTTAAAT TGAAAGCGAG | |
| | AAATAATCAT AAATAATTTC ATTATCGCGA | |
| | TATCCGTTAA GTTTGTATCG TA | |

### DESCRIPTION OF INVENTION

The objective was to improve the growth of recombinant MVA on its producer cells and thus to increase viral yields, particularly in large-scale vaccine production.

The problem was to decrease cytotoxic transgene expression by a recombinant MVA during propagation, while preserving the recombinant MVA's potential to induce transgene-specific immune responses in a vaccine recipient.

To solve this problem, we made use of miRNAs endogenously expressed in avian MVA producer cells for downregulation of transgene expression by recombinant MVA. For this purpose, miRNA target sequences corresponding to miRNAs were inserted into the 3′-UTR region of a transgene.

We followed a strategy to first screen for miRNAs with the highest efficiencies in downmodulating transgene expression by recombinant MVA using a transfected model transgene (EGFP), and then to evaluate selected miRNA target sequences and optimize their use in recombinant MVA.

Here it was demonstrated that the concept of linking miRNA target sequences to transgenes expressed by recombinant MVA is practicable for downregulating the transgenes' expression in MVA producer cells. For the reason alone that vaccinia virus was assumed to mediate an impairment of the cellular microRNA machinery and because of the massive activity of the poxviral promoters used to achieve high amounts of transgene product, this finding could not be expected.

Two heterologous series of miRNA target sequences, each series being arranged in a so-called miRblock, namely miRblock-39 and -42, were selected for the preparation of a modified recombinant MVA-BN-RSV. Criteria for miRblock selection were (i) high activity in mediating downregulation of transgene expression in MVA producer cells, (ii) low sequence similarities amongst the miRNA target sequences in a miRblock, and (Ill) low expression of the related miRNAs in target tissues of vaccination, i.e. blood and skeletal muscles.

Finally, it was demonstrated that MVA-BN-RSV recombinants modified by the insertion of miRNA target sequences (e.g., miRblock-39 and -42) provided higher viral yields during their propagation on MVA producer cells than the non-modified recombinant MVA-BN-RSV. Moreover, in mouse immunization experiments it was shown that miRNA target sequences linked to transgenes in recombinant MVA-BN-RSV did not impair the immunogenicity of RSV derived antigens in vivo.

### Definitions

It must be noted that, as used herein, the singular forms “a”, “an”, and “the”, include plural references unless the context clearly indicates otherwise. Thus, for example, reference to “a nucleic acid sequence” includes one or more nucleic acid sequences.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the appended claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used in the context of an aspect or embodiment in the description of the present invention the term “comprising” can be amended and thus replaced with the term “containing” or “including” or when used herein with the term “having.” Similarly, any of the aforementioned terms (comprising, containing, including, having), whenever used in the context of an aspect or embodiment in the description of the present invention include, by virtue, the terms “consisting of” or “consisting essentially of,” which each denotes specific legal meaning depending on jurisdiction.

When used herein “consisting of” excludes any element, step, or ingredient not specified in the claim element. When used herein, “consisting essentially of” does not exclude materials or steps that do not materially affect the basic and novel characteristics of the claim.

The term “recombinant MVA” as described herein refers to an MVA comprising an exogenous nucleic acid sequence inserted in its genome, which is not naturally present in the wildtype virus. A recombinant MVA thus refers to MVA made by an artificial combination of two or more segments of nucleic acid sequence of synthetic or semisynthetic origin which does not occur in nature or is linked to another nucleic acid in an arrangement not found in nature. The artificial combination is most commonly accomplished by artificial manipulation of isolated segments of nucleic acids, using well-established genetic engineering techniques. Generally, a “recombinant MVA” as described herein refers to MVA that is produced by standard genetic engineering methods, e.g., a recombinant MVA is thus a genetically engineered or a genetically modified MVA. The term “recombinant MVA” thus includes MVA (e.g., MVA-BN®) which has integrated at least one recombinant nucleic acid, preferably in the form of a transcriptional unit, in its genome. Recombinant MVA may express heterologous antigenic determinants, polypeptides or proteins (antigens) upon induction of the regulatory elements e.g., the promoter.

The term “heterologous” as used herein refers to a gene or transgene or DNA sequence that is not native (or is foreign) to the MVA but has been inserted into the MVA artificially using recombinant technologies. Similarly, the expression “heterologous miRblock” refers to a miRblock that is heterologous with respect to the MVA comprising the same.

The term “miRNA” (abbreviation of “microRNA”) refers to a small single-stranded non-coding RNA molecule of typically 21-23 nucleotides (nt) in length. miRNAs function in RNA silencing and post-transcriptional regulation of gene expression by binding to the mRNA.

The nomenclature of miRNAs is principally based on simple sequential numbering of identified miRNAs preceded by the abbreviation for the organism in which the miRNA was identified. All miRNAs referred to herein were identified in CEF cells or chicken tissue and thus the respective miRNA names would have to be preceded by a gga for Gallus gallus (chicken), e.g., the full name of “miR-17-5p” (as used herein) would be gga-miR-17-5p. Since only chicken miRNAs were tested in the study presented herein, we have omitted the gga in all miRNA names for the benefit of legibility.

The term “miRNA sequence” as used herein refers to the mature miRNA nucleotide sequence.

The term “seed sequence” refers to a section within the nucleotide sequence of a miRNA which is essential for the binding between miRNA and mRNA. This section is 7 to 8 nucleotides in length and perfectly complementary to a related section in the mRNA sequence.

The term “miRNA target sequence” as used herein means a nucleic acid sequence corresponding to the nucleotide sequence of a miRNA. The matching between a miRNA sequence and its corresponding target sequence (i.e., nucleotide complementarity) can be 100% fit or less.

The term “corresponding” or “corresponds to” as used herein relates to a nucleotide sequence, e.g. of a miRNA target sequence, with respect to a related nucleotide sequence, e.g. of a miRNA. More precisely, a miRNA target sequence that corresponds to a miRNA represents a counterpart or complement to said miRNA sequence.

The term “complementary” refers to two nucleotide sequences the nucleotides of which match with each other such that the nucleotides can form a double-stranded structure.

The term “miRblock” as used herein refers to a series of miRNA target sequences. A “series” in this context means two, three or more consecutive or concatenated miRNA target sequences.

A “homo-oligomeric” miRblock is composed of a series of identical miRNA target sequences.

A “hetero-oligomeric” miRblock is composed of a series of miRNA target sequences differing in their nucleotide sequences. Usually, all miRNA target sequences in a miRblock differ from each other. Alternatively, two or more miRNA target sequences differ from each other, while others in the miRblock are identical.

The term “downregulation” or “downmodulation” in the context of transgene expression relates to a reduction of or decrease in the amount of a transgene product. This reduction or decrease results from a reduction in the amount of transgene mRNA or from a reduction in the translation of the transgene's mRNA.

A “transcriptional unit” as used herein includes a transgene and promoter operably linked thereto, a terminator and, optionally, a series of miRNA target sequences.

The term “operably linked” as used herein means that a first nucleic acid sequence (e.g., a transgene) is placed in a functional relationship with second nucleic acid sequence (e.g., a promoter). For example, a promoter is operably linked to a coding sequence of a transgene if the promoter is placed in a position where it can direct transcription of the coding sequence.

### ABBREVIATIONS

BFP blue fluorescent protein
bp base pair(s)
CEF chicken embryo fibroblast
CMV cytomegalovirus
DF-1 name of a continuous chicken fibroblast cell line
EGFP enhanced green fluorescent protein
miRb miRblock, i.e. a series of miRNA target sequences
miRNA microRNA
MOI multiplicity of infection
MVA Modified Vaccinia Virus Ankara
MVA-BN MVA-BN® (Bavarian Nordic)
nt nucleotide
ORF open reading frame
p.i. post infection
RFP red fluorescent protein
RSV respiratory syncytial virus
TCID₅₀ tissue culture infection dose 50
TTS transcription termination signal

### EMBODIMENTS

In one embodiment, at least one of the miRNA target sequences in the miRblock is capable of mediating downregulation of the transgene's expression level in the eukaryotic MVA producer cell.

In one embodiment, at least one of the miRNA target sequences in the miRblock mediates a downregulation of the transgene's expression level in a eukaryotic MVA producer cell when bound by a miRNA which the miRNA target sequence corresponds to.

In one embodiment, the downregulation of the transgene's expression level means a lower amount of transgene product, e.g. per cell, as compared to a transgene not linked to any miRNA target sequence.

In one embodiment, the downregulation of the transgene's expression level means a reduction in the level of transgene mRNA or a reduction in translation of the transgene's mRNA.

In one embodiment, the reduction of or decrease in the transgene's expression level relative to the expression level of a transgene not linked to any miRNA target sequence is by about 20, 40, 60, 80, 90, or 99%.

In one embodiment, the series of miRNA target sequences in a miRblock is a series of two, three, four, five, six, seven, eight, or more miRNA target sequences, preferably of three, four, five, six or seven miRNA target sequences, more preferably of three or four target sequences, most preferably of four target sequences.

In one embodiment, the series of miRNA target sequences in a miRblock is a series of from two to ten, preferably of from two to eight miRNA target sequences, more preferably of from three to seven miRNA target sequences, even more preferably of from two to five miRNA target sequences, most preferably of from three to five miRNA target sequences.

In one embodiment, a miRblock is inserted into the 3′-UTR region of the transgene open reading frame (ORF).

In one embodiment, a miRblock is linked to the transgene such that the 5′-first nucleotide of the 5′-first miRNA target sequence is joined to the stop codon of the transgene ORF via a spacer nucleotide sequence of from about 1 to 500 nucleotides or from about 2 to 100 nucleotides or from about 3 to 50 nucleotides, preferably from 5 to 25 nucleotides, more preferably from about 10 to 20 nucleotides, even more preferably from about 13 to 17 nucleotides, most preferably of 15 nucleotides. Alternatively, but less preferred, a miRblock is linked to the transgene such that the 5′-first nucleotide of the 5′-first miRNA target sequence is directly joined to the stop codon of the transgene ORF, i.e. without a spacer nucleotide sequence.

In one embodiment, a transgene operably linked to a poxvirus promoter together with the miRblock linked to the transgene are inserted in an intergenic region (IGR) of the recombinant MVA, preferably selected from the group consisting of IGR 44/45, 64/65, 88/89, and 148/149.

In one embodiment, a transcriptional unit is inserted in an intergenic region (IGR) of the recombinant MVA, preferably selected from the group consisting of IGR 44/45, 64/65, 88/89, and 148/149.

Embodiments Relating to a miRNA Target Sequence

In one embodiment, the miRNA target sequence corresponds to a miRNA such that the miRNA target sequence is capable of binding or partially binding to the miRNA

In one embodiment, the miRNA target sequence which corresponds to a miRNA is partially or completely complementary to the nucleotide sequence of the miRNA.

In one embodiment, at least one miRNA target sequence in a miRblock corresponds to a miRNA sequence at a sequence similarity of from about 80 to 100%, preferably of from about 90 to 100%, more preferably of from about 95 to 100%, most preferably of about 100%. Most preferred is a sequence identity of 100%

In one embodiment, at least one miRNA target sequence in a miRblock comprises a nucleotide sequence outside the seed sequence which corresponds to a miRNA sequence at a sequence similarity of from about 80 to 100%, preferably of from about 90 to 100%, more preferably of from about 95 to 100%, most preferably of about 100%. Most preferred is a sequence identity of 100%.

In one embodiment, at least one miRNA target sequence in a miRblock is complementary to a miRNA sequence, preferably at a sequence similarity of about 100%. Most preferred is a sequence identity of 100%.

In one embodiment, at least one miRNA target sequence in a miRblock is selected from the group consisting of nucleotide sequences as depicted in SEQ ID NO: 1 (corresponding to miR-17-5p), SEQ ID NO: 2 (miR-20a-5p), SEQ ID NO: 3 (miR-21-5p), SEQ ID NO: 4 (miR-221a-3p), SEQ ID NO: 5 (miR-18a-5p), SEQ ID NO: 6 (miR-19a-3p), SEQ ID NO: 7 (miR-199-3p), SEQ ID NO: 8 (miR-33-5p), SEQ ID NO: 9 (miR-218b-5p).

In one embodiment, at least one miRNA target sequence in a miRblock is selected from the group consisting of nucleotide sequences as depicted in SEQ ID NO: 1 (corresponding to miR-17-5p), SEQ ID NO: 2 (miR-20a-5p), SEQ ID NO: 3 (miR-21-5p), SEQ ID NO: 4 (miR-221a-3p), SEQ ID NO: 6 (miR-19a-3p), and SEQ ID NO: 7 (miR-199-3p).

Embodiments Relating to a miRblock

In one embodiment, the series of miRNA target sequences in a miRblock comprises or consists of less than about 200 bp, preferably less than about 150 bp, more preferably about 90 to 100 bp.

In one embodiment, the miRNA target sequences in a miRblock are arranged in a hetero- or homo-oligomeric miRblock, preferably in a hetero-oligomeric miRblock.

In one embodiment, all miRNA target sequences in a hetero-oligomeric miRblock differ from each other. In an alternative embodiment, at least two or most miRNA target sequences in the hetero-oligomeric miRblock differ from each other. Particularly, the miRNA target sequences differ in their nucleotide sequences.

In one embodiment, three or four, preferably four, miRNA target sequences are arranged in a hetero-oligomeric miRblock. Preferably, all three or four, preferably four, miRNA target sequences in the hetero-oligomeric miRblock differ from each other. Particularly, the miRNA target sequences differ in their nucleotide sequences.

In one embodiment, two miRNA target sequences from each of the miRNA target sequences in a miRblock are separated by a spacer nucleotide sequence of from about 1 to 10 nucleotides, preferably from about 2 to 8 nucleotides, more preferably from about 3 to 6 nucleotides, most preferably of 4 nucleotides. Alternatively, but less preferred, the miRNA target sequences in a miRblock are not separated by a spacer nucleotide sequence.

In one embodiment, the miRblock is followed by a poxviral transcription termination signal (TTS).

In one embodiment, the miRNA target sequences in a hetero-oligomeric miRblock are selected from the group consisting of nucleotide sequences as depicted in SEQ ID NO: 1 (corresponding to miR-17-5p), SEQ ID NO: 2 (miR-20a-5p), SEQ ID NO: 3 (miR-21-5p), SEQ ID NO: 4 (miR-221a-3p), SEQ ID NO: 5 (miR-18a-5p), SEQ ID NO: 6 (miR-19a-3p), SEQ ID NO: 7 (miR-199-3p), SEQ ID NO: 8 (miR-33-5p), SEQ ID NO: 9 (miR-218b-5p).

In one embodiment, the miRNA target sequences in a hetero-oligomeric miRblock are selected from the group consisting of nucleotide sequences as depicted in SEQ ID NO: 1 (corresponding to miR-17-5p), SEQ ID NO: 2 (miR-20a-5p), SEQ ID NO: 3 (miR-21-5p), SEQ ID NO: 4 (miR-221a-3p), SEQ ID NO: 6 (miR-19a-3p), and SEQ ID NO: 7 (miR-199-3p).

In one embodiment, a miRblock comprises a nucleotide sequence as depicted in SEQ ID NO: 1 (corresponding to miR-17-5p in miRblock-1).

In one embodiment, a miRblock comprises nucleotide sequences as depicted in SEQ ID NO: 2 (corresponding to miR-20a-5p in miRblock-2), SEQ ID NO: 3 (miR-21-5p-miRblock-2) and SEQ ID NO: 4 (miR-221a-3p-miRblock-2).

In one embodiment, the miRblock comprises nucleotide sequences as depicted in SEQ ID SEQ ID NO: 1 (corresponding to miR-17-5p in miRblock-37), NO: 2 (miR-20a-5p-miRblock-37), SEQ ID NO: 3 (miR-21-5p-miRblock-37), and SEQ ID NO: 6 (miR-19a-3p-miRblock-37).

In one embodiment, the miRblock comprises nucleotide sequences as depicted in SEQ ID NO: 1 (corresponding to miR-17-5p in miRblock-38), SEQ ID NO: 3 (miR-21-5p-miRblock-38), SEQ ID NO: 5 (miR-18a-5p-miRblock-38), and SEQ ID NO: 6 (miR-19a-3p-miRblock-38).

In one embodiment, the miRblock comprises nucleotide sequences as depicted in SE NO: 1 (corresponding to miR-17-5p in miRblock-39), SEQ ID NO: 5 (miR-18a-5p-miRblock-39), SEQ ID and SEQ ID NO: 6 (miR-19a-3p-miRblock-39), and SEQ ID NO: 7 (miR-199-3p-miRblock-39).

In one embodiment, the miRblock comprises nucleotide sequences as depicted in SEQ ID NO: 1 (corresponding to miR-17-5p in miRblock-41), and SEQ ID NO: 4 (miR-221a-3p-miRblock-41), SEQ ID NO: 8 (miR-33-5p-miRblock-41), and SEQ ID NO: 9 (miR-218b-5p-miRblock-41).

In one embodiment, the miRblock comprises or consists of a nucleotide sequence selected from the group consisting of nucleotide sequences as depicted in SEQ ID NO: 48 (corresponding to miRblock-1), SEQ ID NO: 49 (miRblock-2), SEQ ID NO: 55 (miRblock-scrb1-2), SEQ ID NO: 51 (miRblock-37), SEQ ID NO: 52 (miRblock-38), SEQ ID NO: 53 (miRblock-39), and SEQ ID NO: 54 (miRblock-41).

In one embodiment, the miRblock comprises or consists of a nucleotide sequence selected from the group consisting of nucleotide sequences as depicted in SEQ ID NO: 48 (corresponding to miRblock-1), SEQ ID NO: 49 (miRblock-2), SEQ ID NO: 53 (miRblock-39), and SEQ ID NO: 54 (miRblock-41).

In one embodiment, the miRblock comprises or consists of a nucleotide sequence selected from the group consisting of nucleotide sequences as depicted in SEQ ID NO: 53 (corresponding to miRblock-39), and SEQ ID NO: 54 (miRblock-41).

In one embodiment, the miRblock comprises or consists of a nucleotide sequence as depicted in SEQ ID NO: 54 (corresponding to miRblock-41).

### Embodiments Relating to a Promoter

In one embodiment, the promoter is an early/late promoter or an early promoter, preferably an immediate-early promoter.

In one embodiment, the early/late promoter is selected from the group consisting of PrS, Pr7.5 and PrH5m promoters.

In one embodiment, the immediate-early promoter is selected from the group consisting of Pr13.5long, Pr1328, PrLE1 (pHyb) promoters.

In a preferred embodiment, the promoter is a Pr13.5long promoter.

### Embodiments Relating to a Transgene

In one embodiment, the transgene encodes a protein or peptide, preferably a protein or peptide comprising one or more antigenic determinants, more preferably a proteinaceous or peptidic antigen.

In one embodiment, the transgene encodes an antigen selected from the group consisting of a viral, bacterial, fungal, plant, parasite, non-human animal, and human antigen, or an antigenic part thereof.

In one embodiment, the transgene encodes a viral antigen, or a part thereof.

In one embodiment, the viral antigen is derived from a virus selected from the group consisting of alpha-virus, adenovirus, Coxsackievirus, Crimean-Congo hemorrhagic fever virus, cytomegalovirus (CMV), dengue virus, Ebola virus, Epstein-Barr virus (EBV), Eastern, Western or Venezuelan equine encephalitis virus (EEV), Guanarito virus, herpes simplex virus-type 1 (HSV-1), herpes simplex virus-type 2 (HSV-2), human herpesvirus-type 8 (HHV-8), hepatitis A virus (HAV), hepatitis B virus (HBV), hepatitis C virus (HCV), hepatitis D virus (HDV), hepatitis E virus (HEV), human immunodeficiency virus (HIV), influenza virus, Junin virus, Lassa virus, Machupo virus, Marburg virus, measles virus, human metapneumovirus, mumps virus, Norwalk virus, human papillomavirus (HPV), parainfluenza virus, parvovirus, poliovirus, rabies virus, respiratory syncytial virus (RSV), rhinovirus, rotavirus, rubella virus, Sabia virus, severe acute respiratory syndrome virus 2 (SARS-CoV-2), middle east respiratory syndrome coronavirus (MERS-CoV), varicella zoster virus, variola virus, West Nile virus, and yellow fever virus.

In one embodiment, the viral antigen is derived from RSV.

In one embodiment, the transgene encodes a protein derived from RSV, or an antigenic part thereof, preferably selected from the group consisting of RSV G(A), G(B), F, N, and M2-1 protein and a N/M2-1 fusion protein.

In one embodiment, the viral antigen is derived from Eastern, Western or Venezuelan EEV.

In one embodiment, the transgene encodes a protein derived from Eastern, Western or Venezuelan EEV, or an antigenic part thereof, preferably selected from the group consisting of envelope polyproteins E3, E2, 6k, and E1.

In one embodiment, the viral antigen is derived from Epstein-Barr virus.

In one embodiment, the transgene encodes a tumor specific antigen (TSA) or a tumor associated antigen (TAA), or an antigenic part thereof.

Embodiments Relating to a miRNA

In one embodiment, the miRNA in a eukaryotic MVA producer cell is present or detectable or expressed in the eukaryotic MVA producer cell.

In one embodiment, the miRNA is endogenous to a eukaryotic MVA producer cell.

In one embodiment, the miRNA is encoded by, preferably expressed by, a heterologous nucleotide sequence in a transgenic cell line.

In a preferred embodiment, the miRNA is not or only low or moderately expressed in skeletal muscle cells and blood cells such as leucocytes.

### Embodiments Relating to a Eukaryotic MVA Producer Cell

In one embodiment, the eukaryotic MVA producer cell is an avian (e.g., chicken) cell, preferably a primary avian cell or a cell of a permanent avian cell line.

In one embodiment, the eukaryotic MVA producer cell, preferably the primary avian cell, is a chicken embryo fibroblast (CEF) cell.

In one embodiment, the eukaryotic MVA producer cell, preferably the cell of a permanent avian cell line, is a DF-1 or a quail cell.

### Embodiments Relating to MVA

In one embodiment, the recombinant MVA is derived from an MVA or an MVA derivative having the capability of reproductive replication in vitro in chicken embryo fibroblasts (CEF) cells, but no capability of reproductive replication in the human keratinocyte cell line HaCaT, the human bone osteosarcoma cell line 143B, the human embryo kidney cell line 293, and the human cervix adenocarcinoma cell line HeLa.

In one embodiment, the recombinant MVA is derived from MVA-BN® as deposited at the European Collection of Animal Cell cultures (ECACC) under accession number V00083008 on 30 Aug. 2000.

Embodiments Relating to Recombinant MVA with Several Transgenes

In one embodiment, the recombinant MVA comprises more than one, e.g, two, three, four, five, or even more transgenes or transcriptional units.

In one embodiment, the recombinant MVA comprises a first, second and third, or a first to fourth, or a first to fifth, or a first to sixth, or more transcriptional units.

In a preferred embodiment, the recombinant MVA comprises four or a first to fourth transcriptional units.

In one embodiment of the recombinant MVA comprising more than two transgenes, each transgene is different, preferably each transcriptional unit comprises a different transgene.

In one embodiment, the recombinant MVA further comprises one or more transcriptional units not comprising a miRNA target sequence, preferably comprises one or more transcriptional units comprising a nucleotide sequence comprising a transgene operably linked to a poxvirus promoter and no miRNA target sequence linked to the transgene.

Embodiments Relating to Recombinant MVA with Transgenes Encoding RSV Derived Proteins

In one embodiment, the recombinant MVA comprises a transcriptional unit comprising a nucleotide sequence comprising a transgene operably linked to a poxvirus promoter, the nucleotide sequence further comprising a series of miRNA target sequences arranged in a miRblock that is linked to the transgene, wherein each miRNA target sequence corresponds to or is complementary to a miRNA expressed in a eukaryotic MVA producer cell, wherein
(a) the transgene encodes an RSV G(A) protein and, preferably, the poxvirus promoter is a Pr7.5 promoter;
(b) the transgene encodes an RSV G(B) protein and, preferably, the poxvirus promoter is a PrS promoter;
(c) the transgene encodes an RSV F protein and, preferably, the poxvirus promoter is a PrH5m promoter; and/or
(d) the transgene encodes an RSV N/M2-1 fusion protein and, preferably, the poxvirus promoter is a PrLE1 promoter.

In one embodiment, the recombinant MVA comprises a transcriptional unit comprising a nucleotide sequence comprising a transgene operably linked to a poxvirus promoter, the nucleotide sequence further comprising a series of miRNA target sequences arranged in a miRblock that is linked to the transgene, wherein each miRNA target sequence corresponds to or is complementary to a miRNA expressed in a eukaryotic MVA producer cell, wherein the transgene encodes an RSV N/M2-1 fusion protein and the poxvirus promoter is a PrLE1 promoter.

In one embodiment, the recombinant MVA comprises transgenes encoding RSV derived proteins in a first to third transcriptional unit, each transcriptional unit comprising a nucleotide sequence comprising a transgene operably linked to a poxvirus promoter, the nucleotide sequence further comprising a series of miRNA target sequences arranged in a miRblock that is linked to the transgene, wherein each miRNA target sequence corresponds to or is complementary to a miRNA sequence in a eukaryotic MVA producer cell, wherein
(aa) the transgene in the first transcriptional unit encodes an RSV G(A) protein;
(bb) the transgene in the second transcriptional unit encodes an RSV G(B) protein; and
(cc) the transgene in the third transcriptional unit encodes an RSV F protein.

In one embodiment of the recombinant MVA comprising transgenes encoding RSV derived proteins in a first to third transcriptional unit,
(aa′) the miRblock linked to the transgene encoding an RSV G(A) protein comprises or consist of a nucleotide sequence as depicted in SEQ ID NO: 49 (corresponding to miRblock-2);
(bb′) the miRblock linked to the transgene encoding an RSV G(B) protein comprises or consists of a nucleotide sequence as depicted in SEQ ID NO: 48 (miRblock-1); and
(cc′) the miRblock linked to the transgene encoding an RSV F protein comprises or consist of a nucleotide sequence as depicted in SEQ ID NO: 48 (miRblock-1).

In one embodiment, which is a preferred embodiment, the recombinant MVA comprises transgenes encoding RSV derived proteins in a first to fourth transcriptional unit, each transcriptional unit comprising a nucleotide sequence comprising a transgene operably linked to a poxvirus promoter, the nucleotide sequence further comprising a series of miRNA target sequences arranged in a miRblock that is linked to the transgene, wherein each miRNA target sequence corresponds to or is complementary to a miRNA sequence in a eukaryotic MVA producer cell, wherein
(aaa) the transgene in the first transcriptional unit encodes an RSV G(A) protein;
(bbb) the transgene in the second transcriptional unit encodes an RSV G(B) protein;
(ccc) the transgene in the third transcriptional unit encodes an RSV F protein; and
(ddd) the transgene in the fourth transcriptional unit encodes an RSV-N/M2-1 protein.

In one embodiment of the recombinant MVA comprising transgenes encoding RSV derived proteins in a first to fourth transcriptional unit,
(aaa′) the miRblock linked to the transgene encoding an RSV G(A) protein comprises or consist of a nucleotide sequence as depicted in SEQ ID NO: 54 (corresponding to miRblock-41);
(bbb′) the miRblock linked to the transgene encoding an RSV G(B) protein comprises or consists of a nucleotide sequence as depicted in SEQ ID NO: 53 (miRblock-39);
(ccc′) the miRblock linked to the transgene encoding an RSV F protein comprises or consists of a nucleotide sequence as depicted in SEQ ID NO: 53 (miRblock-39); and
(ddd′) the miRblock linked to the transgene encoding an RSV-N/M2-1 protein comprises or consist of a nucleotide sequence as depicted in SEQ ID NO: 54 (miRblock-41).

In one embodiment,
(w) the transgene encodes an RSV G(A) protein, and the poxvirus promoter is a Pr7.5 promoter;
(x) the transgene encodes an RSV G(B) protein, and the poxvirus promoter is a PrS promoter;
(y) the transgene encodes an RSV F protein, and the poxvirus promoter is a PrH5m promoter; and/or
(z) the transgene encodes an RSV N/M2-1 fusion protein, and the poxvirus promoter is a PrLE1 promoter.

In one embodiment, the recombinant MVA comprises a transcriptional unit comprising a nucleotide sequence comprising a transgene operably linked to a poxvirus promoter, the nucleotide sequence further comprising a series of miRNA target sequences arranged in a miRblock that is linked to the transgene, wherein each miRNA target sequence corresponds to or is complementary to a miRNA sequence in a eukaryotic MVA producer cell, wherein the transgene encodes an RSV N/M2-1 fusion protein, the poxvirus promoter is a PrLE1 promoter and the miRblock comprises or consists of a nucleotide sequence as depicted in SEQ ID NO: 54 (miRblock-41).

### Embodiments Relating to Medical Uses or Treatments

In one embodiment, the recombinant MVA for use as a medicament or vaccine. preferably for use in the treatment or prevention of a disease, more preferably for use in the treatment or prevention of an infectious disease or cancer, is for use in a subject.

In one embodiment, the subject is not a bird, preferably is a human or non-human mammal.

In one embodiment, the infectious disease is RSV infection or an infection with Eastern, Western or Venezuelan equine encephalitis virus (EEV) or an infection with Epstein-Barr virus, preferably is RSV infection.

In one embodiment, the recombinant MVA is administered intramuscularly or subcutaneously, preferably intramuscularly.

### Further Embodiments

In one embodiment, the recombinant MVA is propagated on the eukaryotic producer cell at a temperature of from about 30° C. to 37° C., preferably selected from the group of temperatures consisting of about 30° C., 31° C., 32° C., 33° C., 34° C., 35° C., 36° C., and 37° C., most preferably at a temperature of about 30° C. or 37° C.

In one embodiment, the recombinant MVA is propagated in the eukaryotic producer cell at a temperature of about 30° C. or 37° C. in DF-1 cells.

In one embodiment, the recombinant MVA is propagated in a eukaryotic producer cell culture after infection with the recombinant MVA at a multiplicity of infection (MOI) of between 0.001 and 5, preferably at a MOI of 0.001, 0.05, 0.01, 0.05, 0.1, 0.2, 1, 2, or 5.

In one embodiment, the recombinant MVA is propagated in a eukaryotic producer cell culture in a multi-cycle replication setting.

### Further Description

### Modified Vaccinia Virus Ankara (MVA)

In the past, MVA was generated by 516 serial passages on chicken embryo fibroblasts of the Ankara strain of vaccinia virus (CVA) (for review see Mayr et al. 1975). This virus was renamed from CVA to MVA at passage 570 to account for its substantially altered properties. MVA was subjected to further passages up to a passage number of over 570. As a consequence of these long-term passages, the genome of the resulting MVA virus had about 31 kilobases of its genomic sequence deleted and, therefore, was described as highly host cell restricted for replication to avian cells (Meyer et al. 1991). It was shown in a variety of animal models that the resulting MVA was significantly avirulent compared to the fully replication competent starting material (Mayr and Danner 1978).

An MVA useful in the practice of the present invention includes MVA-572 (deposited as ECACC V94012707 on 27 Jan. 1994); MVA-575 (deposited as ECACC V00120707 on 7 Dec. 2000), MVA-1721 (referenced in Suter et al. 2009), NIH clone 1 (deposited as ATCC® PTA-5095 on 27 Mar. 2003) and MVA-BN® (deposited at the European Collection of Cell Cultures (ECACC) under number V00083008 on 30 Aug. 2000).

More preferably the MVA used in accordance with the present invention includes MVA-BN® and MVA-BN® derivatives. MVA-BN® has been described in WO 02/042480. “MVA-BN® derivatives” refer to any virus exhibiting essentially the same replication characteristics as MVA-BN®, as described herein, but exhibiting differences in one or more parts of their genomes.

MVA-BN®, as well as MVA-BN® derivatives, is replication incompetent, meaning a failure to reproductively replicate in vivo and in vitro. More specifically in vitro, MVA-BN® or MVA-BN® derivatives have been described as being capable of reproductive replication in chicken embryo fibroblasts (CEF), but not capable of reproductive replication in the human keratinocyte cell line HaCaT (Boukamp et al 1988), the human bone osteosarcoma cell line 143B (ECACC Deposit No. 91112502), the human embryo kidney cell line 293 (ECACC Deposit No. 85120602), and the human cervix adenocarcinoma cell line HeLa (ATCC Deposit No. CCL-2). Additionally, MVA-BN® or MVA-BN® derivatives have a virus amplification ratio at least two-fold less, more preferably three-fold less than MVA-575 in Hela cells and HaCaT cell lines. Tests and assay for these properties of MVA-BN® and MVA-BN® derivatives are described in WO 02/42480 and WO 03/048184.

The term “not capable of reproductive replication” in human cell lines in vitro as described above is, for example, described in WO 02/42480, which also teaches how to obtain MVA having the desired properties as mentioned above. The term applies to a virus that has a virus amplification ratio in vitro at 4 days after infection of less than 1 using the assays described in WO 02/42480 or U.S. Pat. No. 6,761,893.

### Exemplary Generation of a Recombinant MVA Virus

For the generation of a recombinant MVA as disclosed herein, different methods may be applicable. The DNA sequence to be inserted into the virus can be placed into an E. coli plasmid construct into which DNA homologous to a section of DNA of the poxvirus has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of poxvirus DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA viral DNA in the plasmid and the viral genome, respectively, can generate an MVA modified by the presence of foreign (heterologous) DNA sequences.

A cell of a suitable cell culture as, e.g., CEF cells, can be infected with a MVA virus. The infected cell can be, subsequently, transfected with a first plasmid vector comprising a foreign or heterologous gene or genes, such as one or more of the nucleic acids provided herein, preferably under the transcriptional control of a poxvirus expression control element. As explained above, the plasmid vector also comprises sequences capable of directing the insertion of the exogenous sequence into a selected part of the MVA viral genome. Optionally, the plasmid vector also contains a cassette comprising a marker and/or selection gene operably linked to a poxvirus promoter. The use of selection or marker cassettes simplifies the identification and isolation of the generated recombinant MVA. However, a recombinant poxvirus can also be identified by PCR technology. Subsequently, a further cell can be infected with the recombinant MVA obtained as described above and transfected with a second vector comprising a second foreign or heterologous gene or genes. In case, this gene shall be introduced into a different insertion site of the poxvirus genome, the second vector also differs in the poxvirus-homologous sequences directing the integration of the second foreign gene or genes into the genome of the poxvirus. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection. There are ample of other techniques known to generate recombinant MVA.

The practice of the invention will employ, if not otherwise specified, conventional techniques of immunology, molecular biology, microbiology, cell biology, and recombinant technology, which are all within the skill of the art. See e.g. Sambrook, Fritsch and Maniatis, Molecular Cloning: A Laboratory Manual, 2nd edition, 1989; Current Protocols in Molecular Biology, Ausubel F M, et al., eds, 1987; the series Methods in Enzymology (Academic Press, Inc.); PCR2: A Practical Approach, MacPherson M J, Hams B D, Taylor G R, eds, 1995; Antibodies: A Laboratory Manual, Harlow and Lane, eds, 1988.

### EXAMPLES

The following examples serve to further illustrate the disclosure. They should not be understood as limiting the invention, the scope of which is determined by the appended claims.

### Example 1: Materials and Methods

### 1.1 Cells and Viruses

The chicken fibroblast cell line DF-1 was obtained from ATCC. Primary CEF cells were prepared from 11-day old embryonated chicken eggs. CEF cells were cultured in VP-SFM medium (ThermoFisher Scientific®) supplemented with 1% gentamycin and 4 mM L-glutamine for transfection and virus stock production or DMEM supplemented with 10% FCS for replication analysis and virus titration. The MVA used in this study was derived from a bacterial artificial chromosome (BAC) clone constructed from MVA-BN® (Bavarian Nordic®; herein referred to as “MVA-BN”) and has been described previously (35) (WO 02/42480). MVA-BN® wildtype and MVA-BN recombinants were propagated on CEF or DF-1 cells and titrated on CEF cells using the TCID₅₀ method. Shope fibroma virus for MVA-BAC reactivation was obtained from ATCC (VR-364) and was propagated and titrated on rabbit cornea SIRC cells.

### 1.2 BAC Recombineering and Reactivation of Infectious Recombinant MVA

Construction of the MVA-BACs has been described previously (35). Briefly, the inserted BAC cassette contains miniF plasmid sequences derived from plasmid pMBO131 (36) for maintenance in E. coli. The BAC cassette was inserted between the MVA orthologues of VACV-Copenhagen genes I3L and I4L (MVA064L/MVA065L). The originally contained neomycin-phosphotransferase (npt) II-IRES-EGFP marker cassette was replaced by a bacterial tetracycline expression cassette to remove the enhanced green fluorescence protein (EGFP) gene from the BAC backbone and enable insertion and analysis of an EGFP transgene linked to miRNA target sequences. MVA-BACs were modified by allelic exchange mutagenesis using the counter-selectable rpsL/neo cassette as described (35). The EGFP gene was inserted together with the PrS-gpt-RFP cassette (see FIG. 4) in the intergenic region (IGR) between genes MVA044L/MVA045L (F14L and F15L) in VACV Copenhagen nomenclature under the control of the synthetic poxviral early/late promoter PrS (23). The target sequences for the respective miRNAs (as listed in Table 1 and 2), or miRblock-scrbl2, a control miRblock with a scrambled version of miRblock-2 (see Table 3), were inserted directly following the stop codon in the 3′-UTR of the EGFP gene. Starting with miRblock-13 (for miRblocks, see Table 5 and 6), a 15-nucleotide spacer was inserted between the stop codon of the EGFP ORF and the first nucleotide of the 5′-proximal miRNA target sequence. The poxviral transcription termination sequence for early genes was inserted downstream of the miRNA target sequences. Only the early transcription termination sequence (TTS) was inserted into the 3′-UTR of the EGFP gene in the reference recombinant MVA-EGFP not containing a miRblock (“EGFP expression reference”). For reactivation of infectious virus from MVA-BACs, 10⁶ BHK-21 cells were transfected with 3 μg of BAC DNA using FuGENE® HD transfection reagent (Promega® Corporation, Fitchburg, Wisconsin, US) and 60 min later infected with Shope fibroma virus to provide the necessary helper functions. Reactivated virus was isolated by further passaging in CEF cells and helper virus was eliminated as previously described (35).

Recombinant MVAs containing miRblock-17 and -18 (under the control of the PrS promoter) and the recombinants expressing EGFP-miRblock-2 and EGFP-scrbl2 under the control of the Pr13.5long immediate-early promoter were generated by standard homologous recombination in CEF cells using transfer plasmids with flanking homology regions targeting the transgenes into IGR MVA044L/MVA045L like in the BAC-derived recombinant MVAs described above. The resulting recombinants were purified by three rounds of plaque purification on CEF cells.

All recombinant MVA viral stocks were produced on DF-1 cells and were titrated on CEF cells using the TCID₅₀ method as described (3). Recombinant MVAs for mouse experiments were propagated on CEF cells, purified via two consecutive sucrose cushion centrifugation steps, and titrated on CEF cells using the TCID₅₀ titration method.

1.3 Plasmid Design and Cloning for miRNA Targeted EGFP Expression Analysis

The various miRNA target sequences or miRblocks to be inserted into the 3′-UTR of EGFP were ordered as oligonucleotide primers to serve as reverse PCR primer for the amplification of the EGFP gene together with a forward primer. A DNA fragment with the complete EGFP ORF containing the miRNA target sequences at the 3′-end of the ORF and restriction sites for cloning at both ends was amplified by PCR and cloned into the mammalian expression vector pEGFP-C1 from which the various EGFP genes were expressed under the control of the human CMV promoter active in all mammalian cells as well as in avian cells. The miRNA target sequences that have been tested in the various miRblocks are listed in Table 1 and 2.

### 1.4 Transfection Assay

Freshly prepared CEF cells in VP-SFM medium (4×10⁴ cells/well) and DF-1 cells (3×10⁴ cells/well) in DMEM/10% FCS were seeded on day 0 in 96-well plates. Cells were transfected on day 1 in triplicates with FuGENE® HD Transfection Reagent (Promega® Corporation, Fitchburg, Wisconsin, US) according to manufacturer's instructions using per 96-well 5 μl of transfection mix containing 0.3 μl transfection reagent and 0.1 μg plasmid DNA with a ratio of 1:2 of plasmids expressing either EGFP or BFP driven by the human CMV promoter. Transfection with plasmid encoding EGFP containing no miRblock served as EGFP expression reference, the plasmid encoding EGFP with scrambled miRblock-2 served as control. Subsequently, cells were incubated at 37° C. for time periods as indicated. On day 1 post transfection, cells were trypsinized, washed, and resuspended in PBS-FACS-FORM (1% FCS, 0.1% NaN₃, 1% PFA) and then analyzed for expression of EGFP and BFP by flow cytometry.

### 1.5 Flow Cytometry for Fluorescent Marker Expression Analysis

Cell culture monolayers were washed with PBS and harvested by trypsinization to prepare single cell suspensions. For analysis of EGFP, red fluorescent protein (RFP) and blue fluorescent protein (BFP) expression, cells were resuspended in PBS-FACS (2% FCS, 0.1% NaN₃) and directly analyzed by flow cytometry using an LSR II flow cytometer (BD Biosciences®, Franklin Lakes, New Jersey, US) and FlowJo® software (FlowJo® LLC, Ashland, Oregon, US).

### 1.6 Viral Replication Analysis

For analysis of multicycle virus replication, confluent monolayers in 6-well cell culture plates were infected with sonicated virus dilutions at the indicated multiplicities of infection (MOIs) in 500 μl of DMEM without FCS. After 60 min of adsorption at 37° C. and 5% CO₂, the inoculum was aspirated, cells were washed once with DMEM and were further incubated at 37° C. and 5% CO₂ in DMEM/2% FCS. Cells plus supernatant were harvested at the indicated points in times, freeze-thawed three times and sonicated before titration. MVA yields were determined on CEF cells using the TCID₅₀ titration method as described (3).

### 1.7 Immunoblot Analysis of RSV Protein Expression by Recombinant MVA

Cells were seeded on the day before infection in 12-well tissue culture plates. Infections were performed as previously described (37). At the indicated times after infection cells were washed with cold phosphate-buffered saline (PBS) and lysed in 200 μl 1× Laemmli loading buffer (65 mM Tris-HCl [pH 6.8], 10% glycerol, 2% SDS, 0.1% bromophenol blue, beta-mercaptoethanol [35 μl/ml]) for 5 min at room temperature followed by 3 min sonication and subsequently heated to 95° C. for 5 min. Lysates were centrifuged at 18,000×g for 1 min to remove cell debris. Soluble proteins in cell lysates were separated on precast SDS-polyacrylamide gels (MiniProtean TGX™, 10%, Bio-Rad™ Laboratories, Inc.) and transferred to polyvinylidene difluoride (PVDF) membranes using a Trans-Blot Turbo blotting system (Bio-Rad™ Laboratories, Inc.) and Trans-Blot Turbo transfer packs (Bio-Rad™ Laboratories, Inc.). Membranes were blocked using 5% bovine serum albumin (BSA, Carl Roth GmbH, Karlsruhe, Germany) in Tris-buffered saline (TBS; 50 mM Tris, 150 mM NaCl, pH 7.5) containing 0.1% Tween®-20 detergent and 0.1% NaN₃, and were incubated with the primary antibodies listed below (diluted in blocking buffer) overnight with shaking at 4° C. Membranes were washed between steps with TBS containing 0.1% Tween®-20 four times (20-40 min in total) and incubated for 1 hour with shaking at room temperature with secondary antibodies coupled to horseradish peroxidase and directed against murine or rabbit IgG. The secondary antibodies had been diluted in TBS containing 5% skim milk powder (VWR® International, Delaware, US). Bands were visualized by enhanced chemiluminescence (ECL) using two different substrate reagents, SuperSignal® West Pico (Thermo Fisher Scientific® Inc., Delaware, US) as the standard reagent, and Amersham® ECL Select® Western Blotting Detection Reagent (GE Healthcare™ Life Sciences, Chicago, Illinois, US) in 1:10 dilution for detection with high sensitivity. Signals were recorded using the ChemiDoc® Touch System and Image Lab™ Software (Bio-Rad™ Laboratories, Inc.) for image analysis and quantification.

The following primary antibodies were used in immunoblot analysis: anti-RSV G (acris BM1268), anti-RSF F (abcam ab43812), RSV N (abcam ab94806), or anti-D8 VACV (clone AB12IT-012-001M1) (all mouse, 1:1000).

### 1.8 Mouse Immunization

Groups of 10 female BALB/c mice (Janvier SAS, Saint-Berthevin Cedex, France) were immunized via the intramuscular (i.m.) route with an inoculum of totally 100 μl (50 μl in each hind leg) containing 10⁸ TCID₅₀ of the respective MVA recombinants on days 0 (prime) and 21 (boost). TBS-treated mice (n=5) were included as controls. For analyses, blood was taken via the tail vein at the indicated points in time and collected in PBS containing 2% FCS, 0.1% sodium azide and 2.5 U/ml heparin, and processed as described below. On day 34 after immunization mice were euthanized and splenocytes were prepared for intracellular cytokine staining (ICCS) of T cells. All animal experiments were approved by the government of Upper Bavaria (Regierung von Oberbayern).

### 1.9 T Cell Assays: Dextramer Staining for RSV-M2-1 and MVA-E3-Specific CD8 T Cells

For analysis of RSV- and MVA-specific CD8+ T cells responses, blood was collected from mice on day 7, day 28, and day 34, and peripheral blood mononuclear cells (PBMCs) were prepared by lysing erythrocytes with red blood cell lysing buffer (Sigma-Aldrich®, Germany) according to the manufacturer's instructions. PBMCs were stained with MHC class I dextramers (Immudex® ApS, Denmark) specific for the immunodominant epitopes of RSV M2-1 or MVA E3 (control) in the BALB/c background, as well as for expression of the surface markers CD4, CD8 and CD44.

### 1.10 Intracellular Cytokine Staining and ELISpot Analyses

On day 13 post boost (day 34) mice were killed and spleens were collected to prepare single cell suspensions by collagenase/DNase digestion with mechanically disrupting tissues through a 70-μm cell strainer followed by red blood cell lysis (Sigma-Aldrich®, Germany). For intracellular cytokine staining (ICCS), splenocytes were restimulated for 6 hours with peptides or controls as indicated and thereafter fixed using IC Fixation & Permeabilization Staining kit (eBioscience®, Delaware, US) and stained for expression of cell surface markers and intracellular cytokines IFN-γ, TNF-α, and IL-2. For ELISpot analyses, 5×10⁵ splenocytes/well were restimulated in duplicates with the immunodominant peptides for the indicated RSV proteins and MVA E3 in the H-2d haplotype, and responses were assayed according to manufacturer's protocol (BD™ ELISPOT assay).

### 1.11 ELISA for RSV and MVA-Specific Antibodies

For analysis of RSV- and MVA-specific IgG antibody titers, serum was collected one day before immunization (day −1), on day 20 post prime and day 34 (=day 13 post boost). RSV- and MVA-specific IgG levels in serum were measured by a direct ELISA. 96-well ELISA plates were coated overnight with RSV antigen (Meridian Bioscience®, Inc., Newtown, Ohio, US) or with crude extract from cells infected with MVA-BN®. Samples were titrated using serial dilutions starting at 1:100 for serum. A sheep anti-mouse IgG-HRP (AbD Serotec™) or goat anti-mouse IgG-HRP (Abcam PLC, United Kingdom) was used as detection antibody. The antibody titers were calculated by 4-parameter fit (Magellan® Software) and defined as the serum dilution that resulted in an optical density of 0.24. Geometric mean titers (GMT) and standard errors of the mean (SEM) were calculated using Excel® software (Microsoft® Corporation, Cincinnati, Ohio, US). Antibody titers below the cut-off of the assay (OD<0.24) were given an arbitrary value of “1” for the purpose of calculation.

### 1.12 PRNT for RSV Neutralizing Antibodies

For analysis of RSV plaque reduction neutralization titer, serum was collected one day before immunization (day −1), on day 20 post prime and day 34 (=day 13 post boost). RSV-specific neutralizing titers were measured by plaque reduction neutralization test (PRNT). Two-fold serial dilutions of serum samples were incubated for 30 min with a defined number of RSV-A2 plaque forming units (pfu) to allow for neutralization of the virus. Then, the mixtures were allowed to adsorb on Vero cells for 70 min. Overlay medium was added and plates were incubated for 5 days. After staining with Crystal Violet, PRNT titers were determined and calculated based on the plaque counts using a neural network plaque counting package. The neutralizing titer is indicated as the serum dilution able to neutralize 50% of the mature virus.

### Example 2: Design of miRNA Target Sequences and miRblocks

First of all, miRNAs reported to be most abundant in CEF cells were extracted from the scientific literature (28-34). Additionally, miRNAs in uninfected CEF cells as well as in CEF cells infected with non-recombinant MVA were determined by RNA sequencing.

For a miRNA target sequence, we used the nucleotide sequence exactly matching the nucleotide sequence of a respective miRNA. Usually, four miRNA target sequences were consecutively arranged in a so-called “miRblock” (sometimes abbreviated herein as “miRb”). In some cases, three (or, in one exceptional case, eight) instead of four miRNA target sequences were combined in a miRblock. The composition of a miRblock regarding its individual miRNA target sequences was either hetero- or homo-oligomeric.

The miRNAs selected, their corresponding target sequences and the respectively assembled miRblocks are listed in Tables 5 and 6 below. miRblock-13 to -20 were constructed from miRNA target sequences based on chicken miRNA abundance and specificity data in the literature (25-31). In a few cases (“miR-9999”, “miR-17-5p”) miRNAs were selected from the miRviewer database. miRblock-25 to 36 were constructed from miRNA target sequences identified on the basis of own miRNA sequencing data. miRblock-37 to -47 were composed of miRNA target sequences previously used in miRblock-13 to -36.

TABLE 5
Overview of hetero-oligomeric miRblocks
and the miRNAs they are based on.

| Table 5.1: miRblock-1 to -10 | | Table 5.2: miRblock-37 to -47 | |
|---|---|---|---|
| miRblock | miRNA | miRblock | miRNA |
| miRblock-1 | miR-17-5p | miRblock-37 | miR-20a-5p |
| | miR-103-3p | | miR-21-5p |
| | miR-125b-5p | | miR-17-5p |
| | miR-222a | | miR-19a-3p |
| miRblock-2 | miR-20a-5p | miRblock-38 | miR-18a-5p |
| | miR-21-5p | | miR-21-5p |
| | miR-148a-3p | | miR-17-5p |
| | miR-221-3p | | miR-19a-3p |
| miRblock-scrbl2 | miR-20a-5p-scrbl | miRblock-39 | miR-18a-5p |
| | miR-21-5p-scrbl | | miR-199-3p |
| | miR-148a-3p-scrbl | | miR-17-5p |
| | miR-221-3p-scrbl | | miR-19a-3p |
| miRblock-3 | miR-10a-5p | miRblock-40 | miR-18a-5p |
| | miR-214 | | miR-199-3p |
| | miR-181a-5p | | miR-20a-5p |
| | miR-92-3p | | miR-19a-3p |
| miRblock-5 | miR-1465 | miRblock-41 | miR-33-5p |
| | miR-1559-5p | | miR-218b-5p |
| | miR-1416-5p | | miR-17-5p |
| | miR-1451-5p | | miR-221a-3p |
| miRblock-6 | miR-1720-3p | miRblock-43 | miR-17-5p |
| | miR-1596-5p | (shuffle -39) | miR-18a-5p |
| | miR-1677-3p | | miR-19a-3p |
| | miR-1456-5p | | miR-199-3p |
| miRblock-7 | miR-10000 | miRblock-44 | miR-17-5p |
| | miR-2131-5p | (shuffle -41) | miR-33-5p |
| | miR-9999 | | miR-221a-3p |
| | miR-1677-5p | | miR-218b-5p |
| miRblock-8 | miR-1464 | miRblock-45 | miR-33-5p |
| | miR-1779-3p | | miR-218b-5p |
| | miR-1467-3p | | miR-17-5p |
| | miR-1451-3p | | miR-221a-3p |
| miRblock-9 | miR-1680-3p | | miR-18a-5p |
| | miR-1786 | | miR-199-3p |
| | miR-1684-3p | | miR-148a-3p |
| | miR-6606-5p | | miR-19a-3p |
| miRblock-10 | miR-1662 | miRblock-46 | miR-20a-5p |
| | miR-1434 | | miR-18a-5p |
| | miR-1684-3p | | miR-17-5p |
| | miR-9999 | | miR-19a-3p |
| | | miRblock-47 | miR-20a-5p |
| | | | miR-199-3p |
| | | | miR-17-5p |
| | | | miR-19a-3p |

TABLE 6
Overview of homo-oligomeric miRblocks
and the miRNAs they are based on.

| miRblock | miRNA |
|---|---|
| miRblock-13 | 3x miR-17-5-p |
| miRblock-14 | 3x miR-103-3p |
| miRblock-15 | 3x miR-125b-5p |
| miRblock-16 | 3x-miR-222a |
| miRblock-17 | 4x miR-20a-5p |
| miRblock-18 | 4x miR-21-5p |
| miRblock-19 | 4x miR-148a-3p |
| miRblock-20 | 4x miR-221a-3p |
| miRblock-25 | 4x miR-18a-5p |
| miRblock-26 | 4x miR-19a-3p |
| miRblock-27 | 4x miR-27b-3p |
| miRblock-28 | 4x miR-454-3p |
| miRblock-29 | 4x miR-460a-5p |
| miRblock-30 | 4x miR-239b-5p |
| miRblock-31 | 4x miR-199-3p |
| miRblock-32 | 4x miR-33-5p |
| miRblock-33 | 4x miR-218b-5p |
| miRblock-34 | 4x miR-196-5p |
| miRblock-35 | 4x miR-128-3p |
| miRblock-36 | 4x miR-126-3p |

### Example 3: miRNA Target Sequences in Plasmids

3.1 Construction of Plasmids Containing miRNA Target Sequences

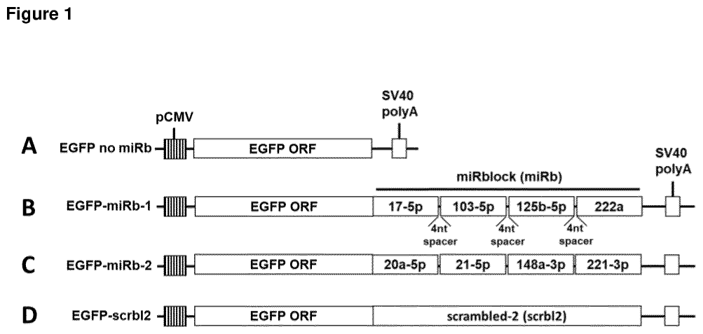

Plasmids were constructed with the objective to assess the potential of miRNA target sequences to mediate downregulation of transgene expression in chicken cells. Inserts of such plasmid constructs are illustrated in FIG. 1.

As a model transgene we used the gene of enhanced green fluorescence protein (EGFP). Expression of the EGFP reporter gene was driven by the standard cytomegalovirus IE promoter (pCMV) (FIG. 1).

The miRNA target sequences in miRblock-1 and -2 (FIGS. 1B and 1C, respectively; “EGFP-miRb-1”, “EGFP-miRb-2”; see also Table 3) were selected because they corresponded to the most abundant miRNAs in CEF cells (25, 27, 30). The four individual miRNA target sequences in miRblock-1 and 2 were separated from each other by 4-nucleotide (nt) spacers. (FIGS. 1B and 1C, respectively). The first nucleotide of the miRNA target sequence at the 5′-end of the miRblock was directly joined to the stop codon of the EGFP open reading frame (ORF). This concept was also applied to miRblock-3 to -10. In all other cases, we inserted a 15-nt spacer between the EGFP stop codon and the first miRNA target sequence.

As a control, we used an EGFP expressing plasmid containing an EGFP ORF without any additionally inserted sequences in the 3′-UTR (“EGFP expression reference”) (FIG. 1A, “EGFP no miRb”). A plasmid containing an EGFP ORF with a miRblock designated “scrambled-2” (or “scrbl2”) in the 3′-UTR served as a further control (FIG. 1D, “scrbl2”). miRblock-scrbl2 was designed by scrambling the nucleotides of each of the four miRNA target sequences of miRblock-2 and joining the scrambled miRNA target sequences again using 4-nt spacers (Table 3). For the scrambling, a tool provided by GeneScript® was used.

### 3.2 Downregulation of EGFP Expression

EGFP downregulating activity of miRblock-1 to -10 was tested.

For this purpose, we co-transfected CEF cells with a plasmid containing the respective miRblock together with a second plasmid expressing blue fluorescent protein (BFP) under the CMV promoter. This BFP expressing plasmid did not contain any additionally inserted sequences in the 3′-UTR encoding region, and it served as a transfection control and internal reference for determining the EGFP expression level in BFP-positive cells.

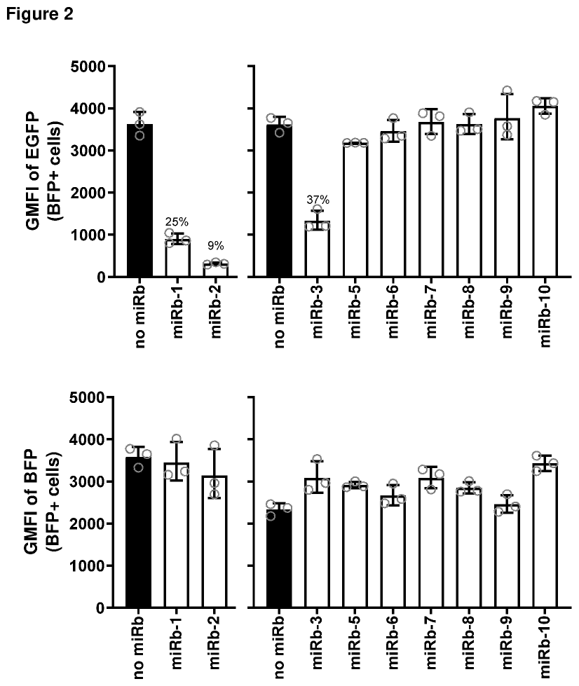

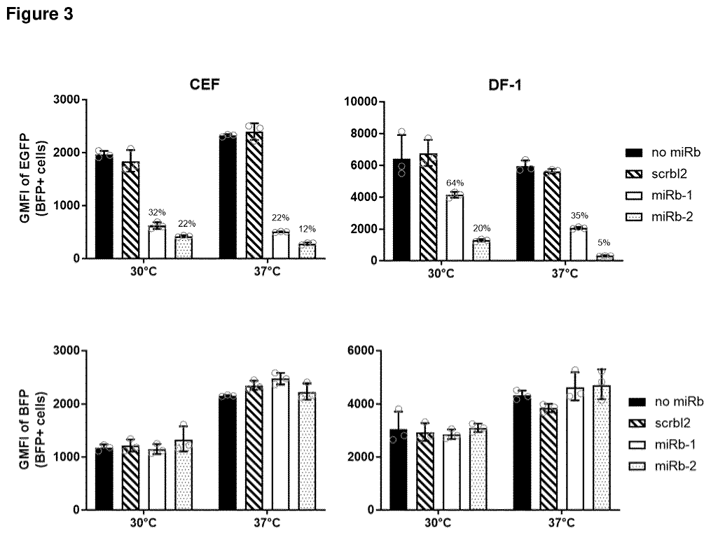

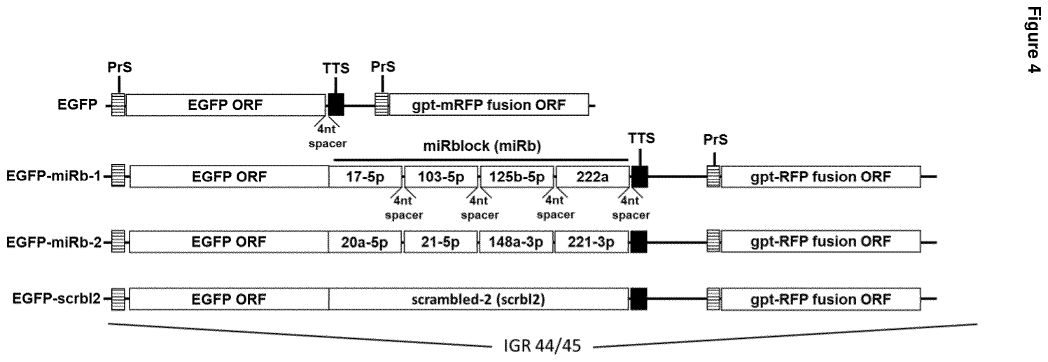

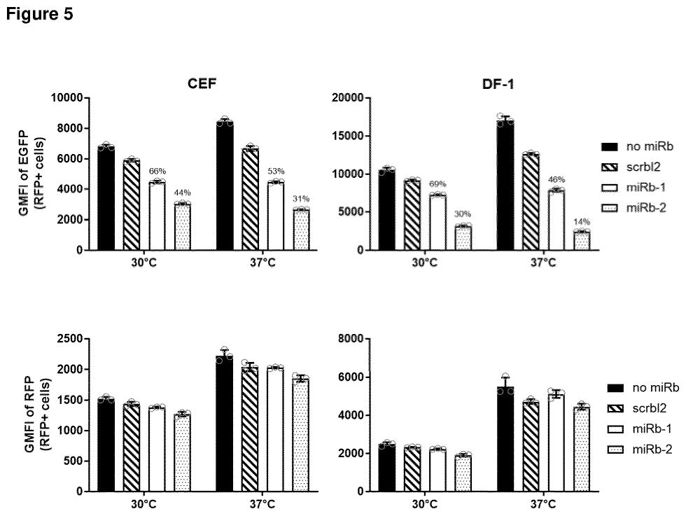

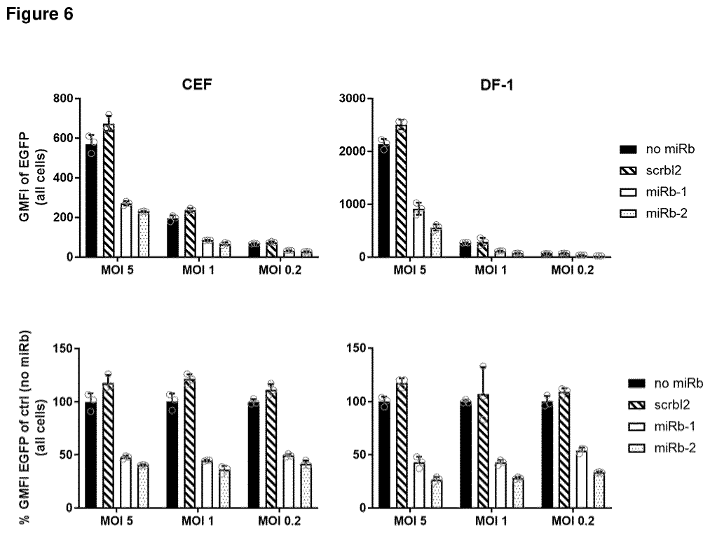

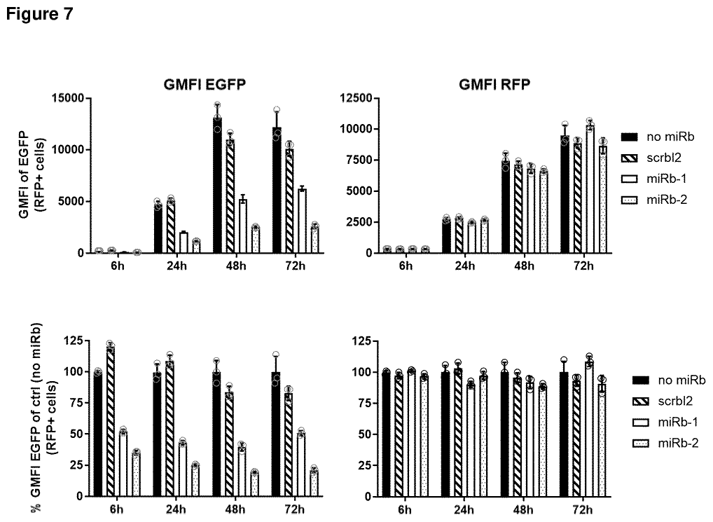

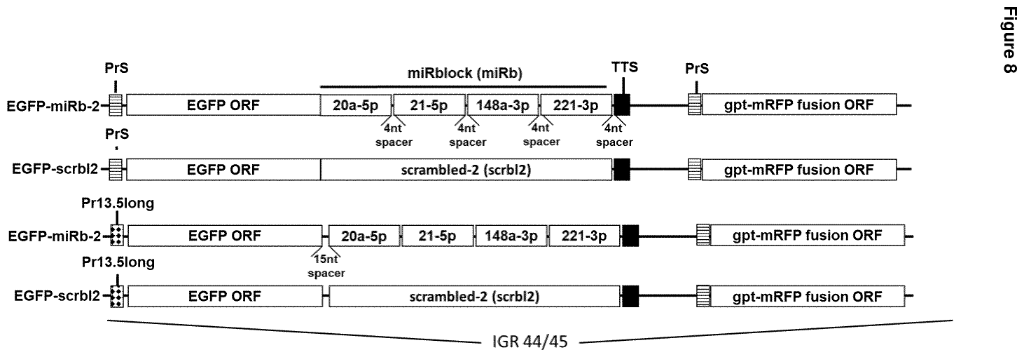

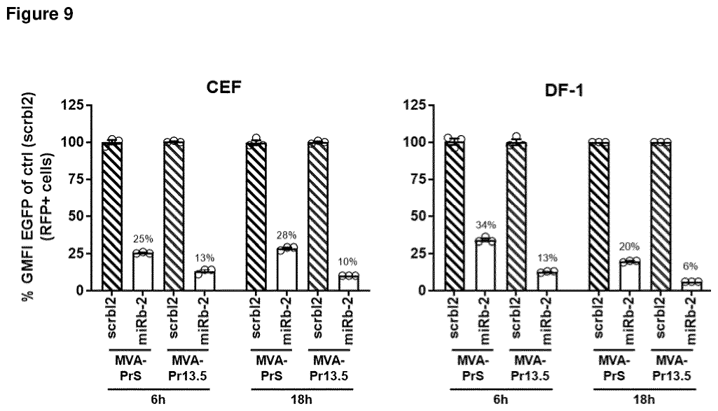

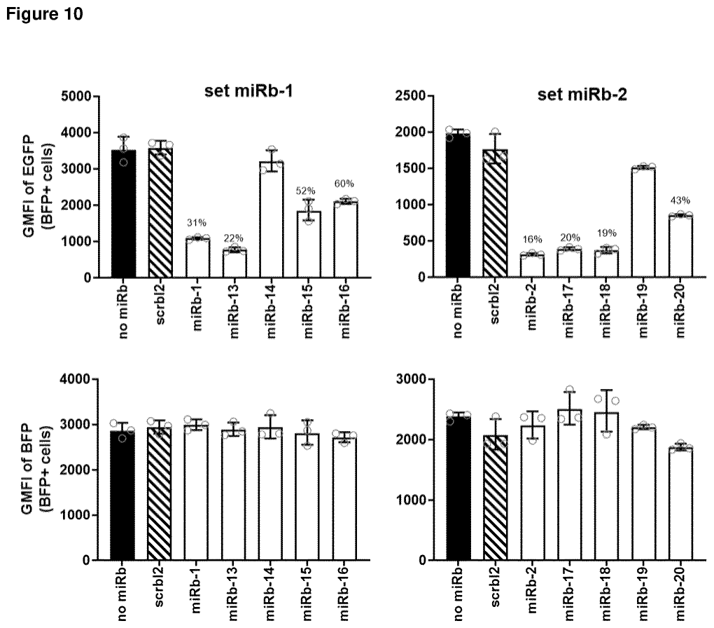

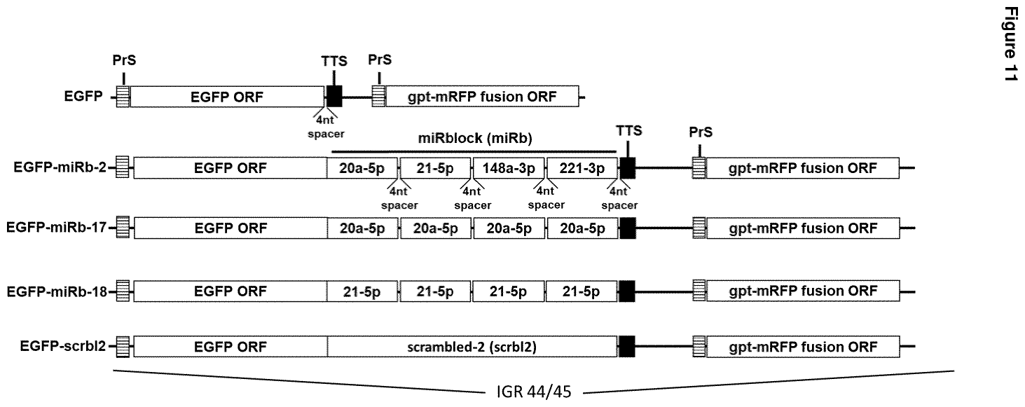

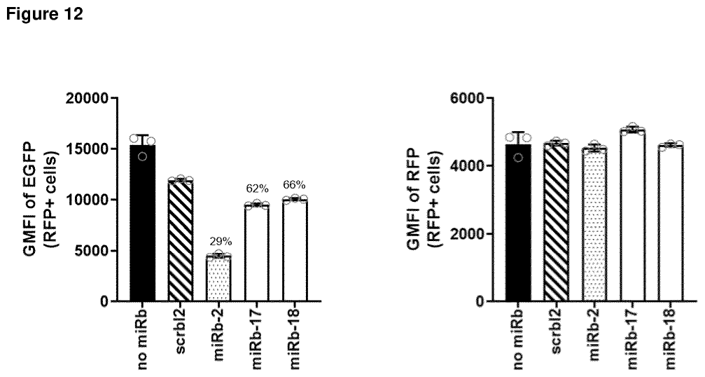

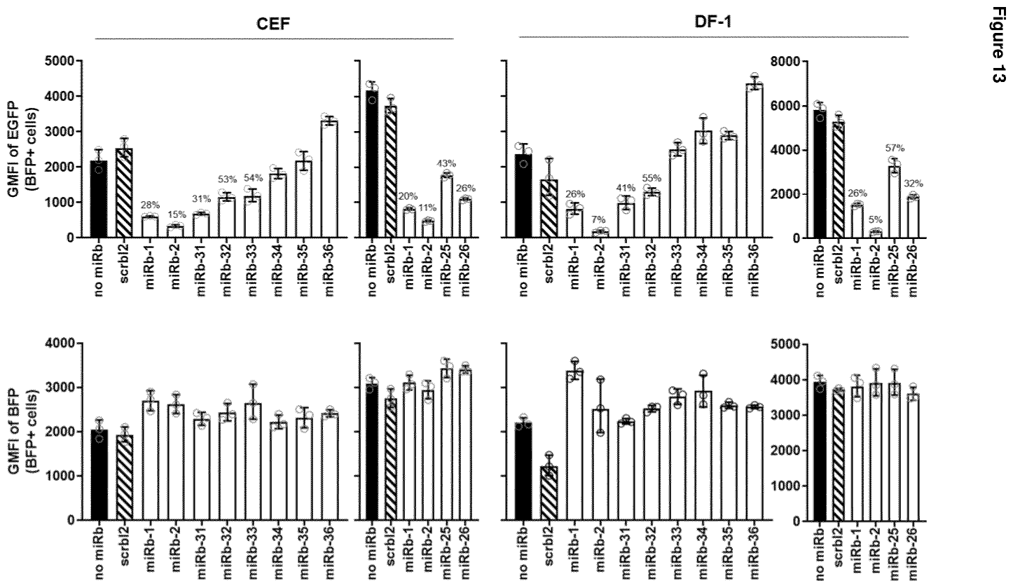

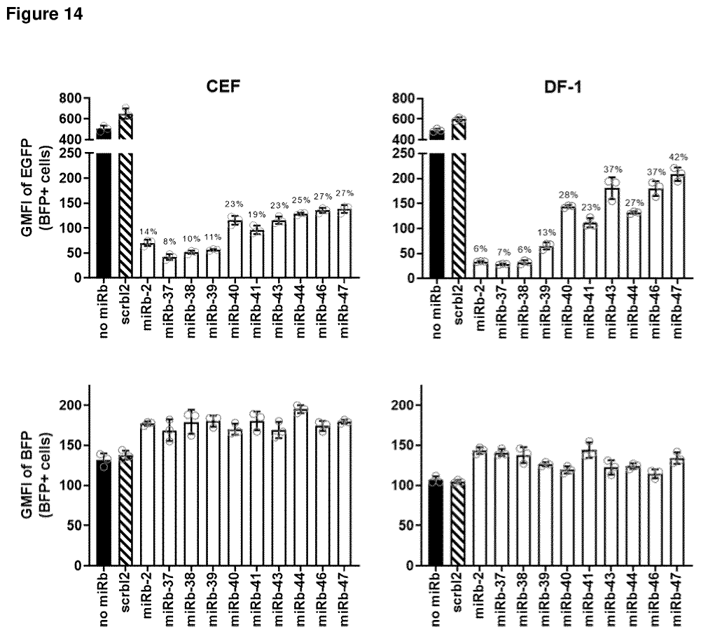

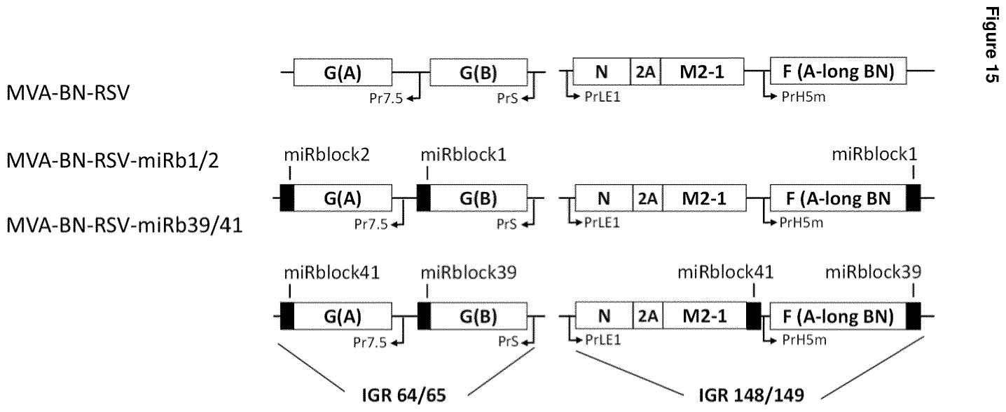

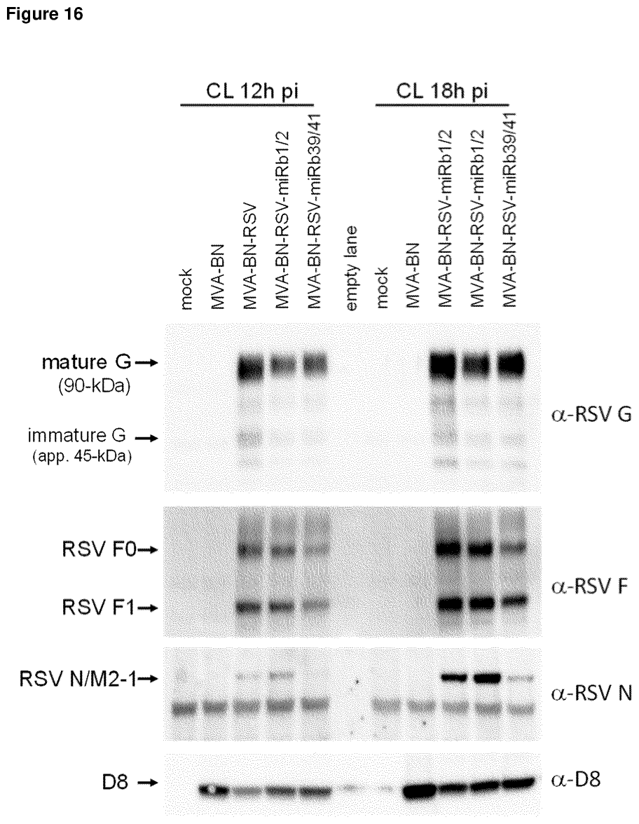

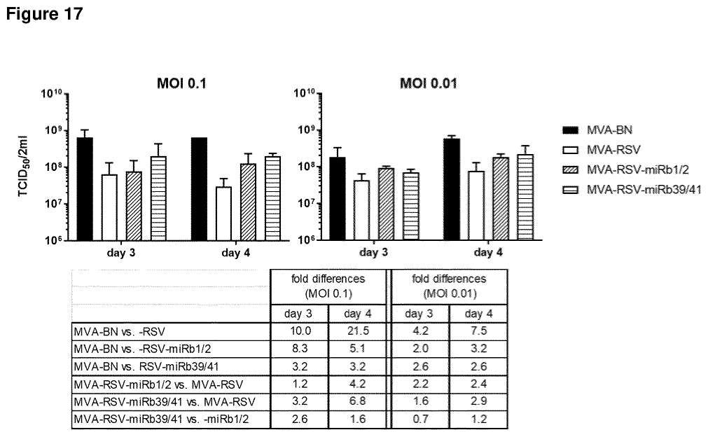

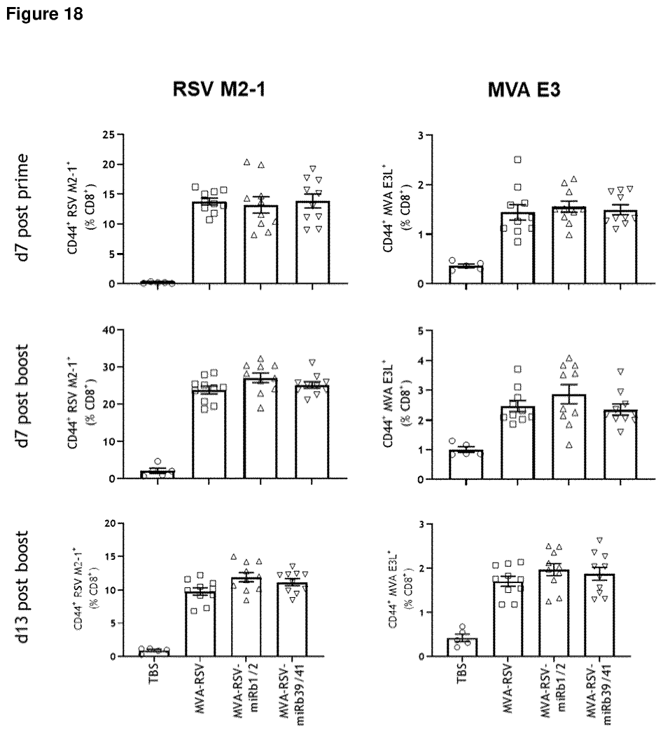

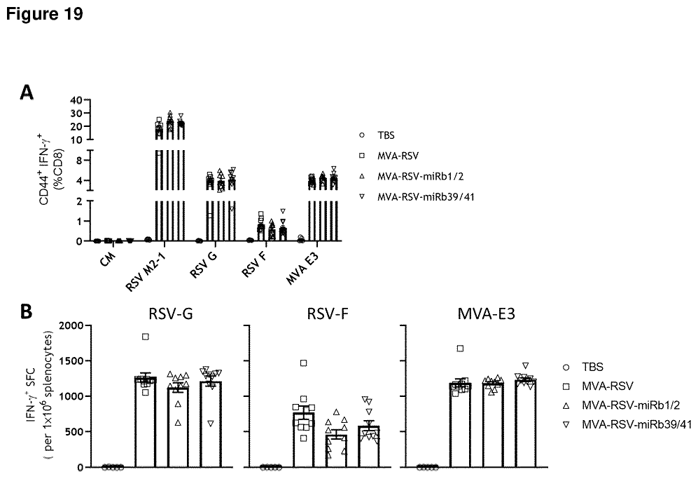

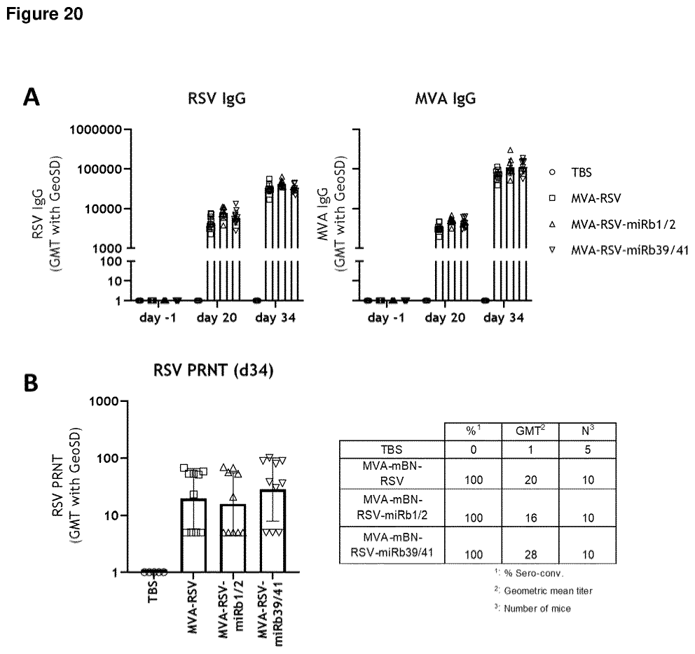

As shown in FIG. 2 (top), miRblock-1 mediated downregulation of EGFP expression to about 25% of control (“no miRb”), and miRblock-2 even mediated EGFP downregulation to about 9% of control. Such effect was not observed for BFP expression by the co-transfected control plasmid (FIG. 2 bottom).

As compared to miRblock-1 and -2, miRblock-3 mediated a moderate EGFP downregulation, while miRblock-5 to -10 showed no significant effect on EGFP expression (FIG. 2 top).

### 3.3 Effect of Temperature on EGFP Downregulation

Based on the results described in Example 3.2, miRblock-1 and -2 were selected for further characterization.

We tested the downregulation of EGFP expression mediated by miRblock-1 and -2 at 30° C. or 37° C. EGFP downregulation in CEF cells was also compared to that in the chicken fibroblast cell line DF-1 using the same constructs and temperatures.

As shown in FIG. 3 (top), the EGFP expression reference (“no miRb”) and the miRblock control (“scrbl2”) yielded similar levels of EGFP expression at a given temperature in both cell types (24 hours after transfection). This finding demonstrated that insertion of a miRblock, i.e., a sequence stretch of approximately 90-100 nucleotides, into the 3′-UTR encoding region of EGFP did not change the transgene's expression level as such, and also that miRblock-scrbl2 was a suitable control.

As furthermore shown, miRblock-1 and -2 mediated downregulation of EGFP expression in CEF and DF-1 cells (FIG. 3 top). In both cell types and at both temperatures the effect produced by miRblock-2 was more pronounced than that produced by miRblock-1.

Moreover, both miRblock-1 and -2 mediated downregulation of EGFP expression more effectively at 37° C. as compared to 30° C. This temperature effect was more pronounced in DF-1 cells (FIG. 3 top).

BFP expression by the co-infected control plasmid was again independent of EGFP downregulation (FIG. 3 bottom).

### Example 4: miRNA Target Sequences in Recombinant MVA

4.1 Construction of Recombinant MVA Containing miRNA Target Sequences

Next, it was examined whether EGFP downregulation could also be achieved in the context of MVA infection using miRNA target sequences.

For that purpose, we constructed two MVA recombinants having inserted in intergenic region (IGR) 44/45 an EGFP gene linked to miRblock-1 or miRblock-2 (FIG. 4, “EGFP-miRb-1, “EGFP-miRb-2”). Each miRblock was followed by a poxviral early transcription termination signal (TTS) serving to limit the length of early transcripts to about 50 nucleotides. The TTS is a standard element in gene expression cassettes for poxviral vectors. Monomeric red fluorescent protein (RFP) was co-expressed by all MVA recombinants (FIG. 4). The RFP gene had not been modified by insertion of miRNA target sequences and should therefore not be regulated by the miRNA machinery of the infected cell. It was inserted to serve as a marker to monitor and compare infection levels produced by the different MVA constructs. Both EGFP and RFP were under control of the early/late PrS promoter in the recombinant MVA constructs (FIG. 4).

As controls, we constructed a recombinant MVA containing the EGFP ORF without miRNA target sequences (FIG. 4, “EGFP”), i.e. an EGFP expression reference, and a recombinant MVA with miRblock-scrbl2 in the 3′-UTR of EGFP (FIG. 4, “EGFP-scrbl2”).

### 4.2 Downregulation of EGFP Expression and Effect of Temperature

We then analyzed the EGFP downregulation mediating activity of miRblock-1 and -2 in cells infected with the respective MVA recombinant and cultured at 30° C. or 37° C.

As shown in FIG. 5 (top), EGFP expression by recombinant MVA was downregulated by miRblock-1 and -2 both in CEF and DF-1 cells. Similar to the observations with plasmids, miRblock-2 was more effective, in particular in DF-1 cells.

Furthermore, EGFP downregulation was detectable at both temperatures, but was more pronounced at 37° C. than at 30° C. in both cell types, and this temperature effect was more pronounced in DF-1 cells (FIG. 5 top).

Co-expression of RFP was independent of EGFP downregulation (FIG. 5 bottom). This demonstrated that expression of an MVA transgene (here: EGFP gene) can be modulated selectively by miRNA target sequences without concurrently affecting other transgenes (here: RFP gene) present in the same recombinant MVA.

### 4.3 Effect of the MOI on EGFP Downregulation

In order to test whether the multiplicity of infection (MOI) affected EGFP downregulation by miRblock-1 or -2, cells were infected with the respective MVA recombinant at MOI 5, 1, or 0.2.

As shown in FIG. 6 (top), downregulation of EGFP expression mediated by miRblock-1 and -2 was detectable at 6 hours p.i. at all MOIs tested. Notably, when EGFP expression is depicted as % of control (i.e., EGFP expression reference, “no miRb”) (FIG. 6 bottom) it becomes apparent that the downregulating effect of miRblock-1 and -2 is independent of the MOI used. Thus, a higher EGFP expression level in an infected cell culture due to a higher MOI did not affect downregulation of EGFP expression.

### 4.4 Effect of Multi-Cycle MVA Replication on EGFP Downregulation

Transgene downregulation was further examined in a setting similar to that usually applied during virus propagation for the production of viral stocks or vaccine lots. For that purpose, CEF cells were infected with recombinant MVA containing miRblock-1 or -2 at a low MOI of 0.1 and cultured over a prolonged incubation period of 3 days.

As shown in FIG. 7 (top, left), the EGFP expression level increased over time (at least from 6 to 48 hours p.i.). However, increases in EGFP expression after infection with recombinant MVA containing miRblock-1 or -2 were significantly reduced compared to those observed for the EGFP expression reference (“no miRb”) and the miRblock control (“scrbl2”).

When EGFP expression was depicted as % of control (“no miRb”) (FIG. 7 bottom, left) it turned out that EGFP downregulation by miRblock-1 and -2 remained largely constant over the whole observation period of 72 hours p.i.miRblock-2 was more effective in mediating downregulation of EGFP expression than miRblock-1.

Levels of RFP co-expressed by the recombinant MVAs also increased over time (up to 72 hours p.i.) (FIG. 7 top, left), but without a downregulation being observed like for EGFP (FIG. 7 top, right and bottom, right), indicating similar infection efficiencies and viral spread through the cell cultures.

Thus, the efficiency of EGFP downregulation mediated by miRblock-1 and -2 remained stable over the course of multi-cycle MVA replication.

### 4.5 Effect of the Poxvirus Promoter

Poxviral promoters used to drive transgene expression have frequently been chosen from a class of combined promoters that initiate expression in the early as well as late phase of viral infection. The synthetic PrS promoter, designed to induce strong transgene expression (23) is a classic example thereof and is widely used.

Despite the presence of an optimized early promoter motif in the PrS promoter, transgene expression under the control of this promoter occurs predominantly late. However, for best possible induction of T cell responses against a transgene product, early and even immediate-early expression of the transgene is favorable.

Therefore, we performed an experiment in which the PrS promoter used so far (see, e.g., FIG. 4) was replaced with the immediate-early Pr13.5long promoter (24) (WO 2014/063832). EGFP expression under the control of the PrS or Pr13.5long promoter was compared in recombinant MVAs containing miRblock-2 in the 3′-UTR encoding region of EGFP (FIG. 8). Recombinant MVAs with miRblock-scrbl2 and either the PrS or Pr13.5long promoter were used as controls (FIG. 8).

As shown in FIG. 9, EGFP expression directed by both the PrS and Pr13.5 promoter was downregulated by miRblock-2 in CEF and DF-1 cells at 6 hours and 18 hours p.i. Notably however, the downregulation of Pr13.5long-driven EGFP expression was significantly more effective as compared to the PrS-driven EGFP expression, namely in both cell types and at both points in time (FIG. 9).

### Example 5: Evaluation of miRNA Target Sequences Using Homo- and Hetero-Oligomeric miRblocks

5.1 EGFP Downregulation by Individual miRNA Target Sequences in Plasmids

In order to determine the contribution of an individual miRNA target sequence to EGFP downregulation mediated by a hetero-oligomeric miRblock, we generated plasmid expression constructs with homo-oligomeric repeats of individual miRNA target sequences previously arranged in a hetero-oligomeric miRblock.

We first examined the eight different target sequences of miRblock-1 and -2. Each miRNA target sequence contained in miRblock-1 was arranged in three identical copies (triplicate repeat) in a homo-oligomeric miRblock (see Table 6, miRblock-13 to -16). Each miRNA target sequence contained in miRblock-2 was arranged in four copies (quadruple repeat) (see Table 6, miRblock-17 to -20). Using the target sequence for miR-125b-5p (contained in miRblock-1) as an example we had observed that EGFP downregulation was not significantly different with either three or four copies arranged in a homo-oligomeric miRblock. Within a homo-oligomeric miRblock the individual miRNA target sequences were separated by 4-nt spacers as described above for hetero-oligomeric miRblocks (see Example 3.1).

As shown in FIG. 10 (top), miRblock-13, -17 and -18 (composed of target sequences for miR-17-5p, miR-20a-5p and miR-21-5p, respectively), were most efficient amongst homo-oligomeric miRblock-13 to -20 in downregulating EGFP expression. The extent of EGFP downregulation mediated by miRblock-13, -17 and -18 in % of control (“no miRb”) was in the range of the hetero-oligomeric miRblock-1 and -2 mediated effect (FIG. 10 top). miRblock-15, -16 and -20 (target sequences for miR-125b-5p, miR-222a and miR-221-3p, respectively) mediated EGFP downregulation at a medium level, while mRblock-14 and -19 (target sequences for miRNAs 103-3p and 148a-3p, respectively) had only minor effects (FIG. 10 top).

BFP expression was very similar in all transfected CEF cultures confirming similar transfection efficiencies of the plasmid constructs (FIG. 10 bottom).

The results obtained were interesting insofar as miR-222a and miR-221a-3p (miRblock-16 and -20, respectively) had been described as the most abundant miRNAs in CEF cells (28). In our experiment however, their corresponding target sequences mediated only moderate downregulation of EGFP expression (FIG. 10 top). On the other hand, abundance of miR-17-5p and miR-20a-5p (miRblock-13 and -17, respectively) was reported to be only approximately 10% of that of miR-222a and miR-221-3p (28), while in our experiment their corresponding target sequences turned out to be even more effective (FIG. 10 top).

5.2 EGFP Downregulation by Selected miRNA Target Sequences in Recombinant MVA

As described above in Example 5.1 and shown in FIG. 10, homo-oligomeric miRblock-13, -17 and -18 were particularly effective in mediating downregulation of EGFP expression by plasmid transfection.

The efficiency of EGFP downregulation by miRblock-17 and -18 was next determined in the context of MVA infection. Two recombinant MVAs were constructed that expressed EGFP under the PrS promoter and contained either miRblock-17 or miRblock-18 in the 3′-UTR encoding region of EGFP (FIG. 11, “EGFP-miRb-17” and “EGFP-miRb-18”, respectively). For comparison, recombinant MVA containing miRblock-2 was used (FIG. 11, “EGFP-miRb-2”). The miRNA target sequences which miRblock-17 and -18 were composed of were also contained in hetero-oligomeric miRblock-2.

As shown in FIG. 12 (left), EGFP expression in CEF cells was downregulated by miRblock-2 to about 29% of control (“no miRb”). In the case of the MVA recombinants containing miRblock-17 or -18, EGFP expression was decreased to only about 62% and 66%, respectively (FIG. 12 left).

Expression of RFP by the different MVA recombinants was very similar (FIG. 12 right), indicating similar levels of MVA infection.

It was remarkable that miRblock-17 and -18 achieved an extent of downregulation of EGFP expression of only about half of that produced by miRblock-2 (FIG. 12 left). This result differed from that obtained with plasmids. As demonstrated above (Example 5.1, see also FIG. 10), EGFP downregulation by miRblock-17 and -18 when expressed from plasmids was quite similar with that produced by miRblock-2. It thus appears that the arrangement of miRNA target sequences in hetero-oligomeric miRblocks may be particularly advantageous when applied in MVA recombinants.

5.3 Screening of Further Potential miRNA Target Sequences in Plasmids

We conducted an own RNA sequencing analysis of small RNAs contained in CEF cells. EGFP downregulation by miRNAs resulting from this analysis was tested.

The target sequences for the selected miRNAs were arranged in homo-oligomeric miRblocks in quadruple repeats separated by 4-nt spacers (see Table 6, miRblock-25 to -36). The miRblocks were placed into the sequence stretch of the EGFP ORF, and EGFP was expressed by plasmid transfection.

As shown in FIG. 13 (top), miRblock-26 and -31 (target sequences for miR-19a-3p and miR-199-3p, respectively) were most efficient amongst miRblock-25 to -36 in downregulating EGFP expression in CEF and DF-1 cells. miRblock-25 and -32 (target sequences for miR-18a-5p and miR-33-5p, respectively) mediated EGFP downregulation at a medium level. The similar applied to miRblock-33 (target sequences for miR-218b-5p), at least in CEF cells. In contrast, remaining miRblock-27 to -30 and miRblock-33 to -36 showed only minor or no downregulating activity, or even an enhancing effect on EGFP expression was observed (FIG. 13 top).

Similar levels of RFP expression in CEF and DF-1 cells confirmed equal transfection efficiencies (FIG. 13 below).

5.4 Defining miRNAs Most Suitable for Downregulating EGFP Expression by Recombinant MVA

The knowledge gained about EGFP downregulating activity of different miRNA target sequences (see Examples 5.1, 5.2 and 5.3) served as a basis for optimizing the design of hetero-oligomeric miRblocks for use in recombinant MVA. The miRNAs selected to this effect are listed in Table 7 below.

TABLE 7
miRNA target sequences used for hetero-
oligomeric miRblock optimization and
corresponding homo-oligomeric
miRblocks

| | | miRNA | SEQ ID |
|---|---|---|---|
| miRblock | miRNA | target sequence | NO: |
| miRblock-13 | miR-17-5p | AGACTACCTGCACTG | SEQ ID |
| | | TAAGCACTTTG | NO: 1 |
| miRblock-17 | miR-20a-5p | CTACCTGCACTATAA | SEQ ID |
| | | GCACTTTA | NO: 2 |
| miRblock-18 | miR-21-5p | TCAACATCAGTCTGA | SEQ ID |
| | | TAAGCTA | NO: 3 |
| miRblock-20 | miR-221a-3p | GAAACCCAGCAGACA | SEQ ID |
| | | ATGTAGCT | NO: 4 |
| miRblock-25 | miR-18a-5p | CTATCTGCACTAGAT | SEQ ID |
| | | GCACCTTA | NO: 5 |
| miRblock-26 | miR-19a-3p | TCAGTTTTGCATAGA | SEQ ID |
| | | TTTGCACA | NO: 6 |
| miRblock-31 | miR-199-3p | CCAATGTGCAGACTA | SEQ ID |
| | | CTGTA | NO: 7 |
| miiRblock-32 | miR-33-5p | GCAATGCAACTACAA | SEQ ID |
| | | TGCAC | NO: 8 |
| miRblock-33 | miR-218b-5p | ACATGGTTAGATCAA | SEQ ID |
| | | GCACAA | NO: 9 |

Repeats of nucleotide sequences generally bear the risk of homologous recombination during MVA replication. Thus, partly or completely homo-oligomeric repeats of miRNA target sequences in miRblocks might lead to unwanted deletions or rearrangements in the newly generated recombinant MVA genome. The probability for such events is assumingly highest when identical sequences or sequence stretches with high similarity are in a tandem arrangement and thus closely or directly adjacent to each other.

Therefore, we aimed at designing hetero-oligomeric miRblocks, the miRNAs of which being maximally different with regard to their nucleotide sequence. As a result, hetero-oligomeric miRblock-37 to -47 were generated (Table 5). miRblock-43 and -44 contained the same miRNA target sequences as miRblock-39 and -41, respectively, but in a different order.

EGFP downregulation was analyzed in plasmids containing one of miRblock-37 to -47 were joined to the EGFP encoding sequence as already described above.

As shown in FIG. 14 (top, left), miRblock-37, -38 and -39 mediated EGFP downregulation most effectively amongst miRblock-37 to -47 in CEF cells. The extent of downregulation of EGFP expression (in % of control, “no miRb”) was even better than that produced by miRblock-2. In DF-1 cells (FIG. 14 top, right), EGFP downregulation at least by miRblock-37 and -38 was in the range of that induced by miRblock-2. Remaining miRblock-40 to -47 mediated downregulation of EGFP expression less effectively (FIG. 14 top).

Expression of RFP by the different MVA recombinants was again very similar (FIG. 14 below).

A recombinant MVA containing a hetero-oligomeric miRblock with eight different miRNA target sequences (miRblock-45 in Table 4) was also tested but showed only little activity in EGFP downregulation.

From the miRblocks showing best EGFP downregulation activity, lead candidate miRblocks were selected based on the following considerations: Firstly, miRNA target sequences with a high or very high expression in human blood, leukocytes or skeletal muscle were avoided. The reason behind was that we considered intramuscular or subcutaneous application as the most common route of vaccination. Data for miRNA expression in human blood, leukocytes and skeletal muscle were taken from the miRgeneDB database (http://mirgenedb.org/). Because miRNA-21-5p is highly expressed in human leukocytes, miRblocks containing the corresponding target sequence, i.e., miRblock-37 and -38, were excluded from further consideration. Secondly, miRblocks containing the target sequences for both miR-17-5p and miR-20a-5p, i.e., miRblock-37, -46 and -47, were not considered further due to the high sequence similarity of these two target sequences.

Finally, miRblock-39 and -41 meeting the miRNA target sequence exclusion criteria and having an EGFP downregulation activity nearest to that of miRblock-37 and -38 were identified as most suitable for the application in recombinant MVA.

### Example 6: miRNA Target Sequences in Recombinant MVA-BN-RSV

6.1 Construction of MVA-BN-RSV Modified by miRNA Target Sequences

We examined the effect of miRNA target sequences on the expression of transgenes present in MVA-BN®-RSV (referred to as “MVA-BN-RSV”) (WO 2014/019718).

MVA-BN-RSV encodes five proteins of human respiratory syncytial virus (RSV) (FIG. 15). The two ORFs of the glycoprotein (G) from both circulating RSV antigenic subtypes A and B are expressed under the control of early/late promoters Pr7.5 (G(A)) and PrS (G(B)), respectively. The fusion glycoprotein (F) gene is expressed under the control of the PrH5m early/late promoter. The nucleoprotein (N) and M2-1 proteins, serving primarily as targets for the antiviral CD8 T cell response, are encoded as fusion protein N/M2-1, and the respective ORF is expressed under the control of the immediate-early promoter PrLE1 (FIG. 15) ((35), PrLE1 is referred to as pHyb in said reference).

A first version of modified MVA-BN-RSV containing miRblock-1 and -2 (“MVA-BN-RSV-miRb1/2” in FIG. 15) was generated aiming at downregulating the expression of RSV glycoproteins G(A), G(B) and F. miRblock-1 was inserted in the 3′-UTR region of the G(B) gene and the F gene, while miRblock-2 was inserted in the 3′-UTR region of the G(A) gene (FIG. 15).

In a second version of modified MVA-BN-RSV (“MVA-BN-RSV-miRb39/41” in FIG. 15), all RSV-derived transgenes were linked to miRNA target sequences. MVA-BN-RSV-miRb39/41 was designed and generated using those miRblocks, i.e., miRblock-39 and -41, which have been identified as most suitable for transgene downregulation in recombinant MVA (see Example 5.4).

### 6.2 Transgene Downregulation in Modified MVA-BN-RSV

Expression of RSV G, F and N/M2-1 proteins in CEF cells infected with MVA-BN-RSV-miRb1/2 or MVA-BN-RSV-miRb39/41 was analyzed by immunoblot and compared to the respective protein expression in MVA-BN-RSV.

In lysates from CEF cells infected with MVA-BN-RSV, MVA-BN-RSV-miRb1/2 or MVA-BN-RSV-miRb39/41 for 12 or 18 hours, RSV G was detected predominantly in its fully glycosylated mature form having a molecular weight of approximately 90 kDa (FIG. 16). In this respect it should be noted that the antibody used for RSV G detection did not discriminate between the two antigenic subtypes A and B of the RSV G protein. Thus, the RSV G-specific signal seen in the immunoblot of FIG. 16 is likely composed of superposed signals for RSV G(A) and G(B). Compared to cells infected with the MVA-BN-RSV control, expression levels of both mature and immature RSV G were lower in cells infected with MVA-BN-RSV-miRb1/2 or MVA-BN-RSV-miRb39/41 (FIG. 16). Thus, miRblock-1 and -2 (linked to RSV G(B) and (G)A in MVA-BN-RSV-miRb1/2, respectively) as well as miRblock-39 and -41 (linked to RSV G(B) and G(A) in MVA-BN-RSV-miRb39/41, respectively) mediated downregulation of RSV G(A)/(B) protein expression.

The RSV F protein was detectable as precursor protein F0 and the large F1 subunit (generated by proteolytic cleavage of F0 by the cellular furin protease) (FIG. 16). The expression of RSV F0/F1 by MVA-BN-RSV-miRb1/2 was only moderately reduced as compared to the MVA-BN-RSV control but was clearly reduced in the case of MVA-BN-RSV-miRb39/41 (FIG. 16). Thus, miRblock-39 was more effective in downregulating RSV F0/F1 expression than miRblock-1.

Expression of the RSV N/M2-1 fusion protein (approximately 62 kDa) in MVA-BN-RSV-miRb39/41 infected cells was clearly reduced as compared to cells infected with the MVA-BN-RSV control or with MVA-BN-RSV-miRb1/2 (FIG. 16). It should be noted in this respect, that MVA-BN-RSV-miRb1/2 did not contain any miRNA target sequences in the RSV N/M2-1 ORF (see FIG. 15).

The expression of vaccinia virus D8 protein used as an endogenous expression control was comparable in cells infected with MVA-BN-RSV-miRb1/2 or MVA-BN-RSV-miRb39/41 (FIG. 16). Interestingly, the D8 signal was slightly stronger in lysates from cells infected with wildtype, i.e. non-recombinant MVA-BN® (FIG. 16) as compared to the three recombinants. This result might indicate that the cytotoxic effect of simultaneously expressed RSV derived transgenes without any miRNA-mediated downregulation affected the expression of autochthonous MVA genes such as D8, while on the other hand, miRNA-targeting of the RSV transgenes did not fully restore normal D8 expression.

Taken together, expression of RSV G(A)/(B) and RSV F0/F1 is downregulated in MVA-BN-RSV-miRb1/2 and MVA-BN-RSV-miRb39/41. However, downregulation of RSV F0/F1 expression is more pronounced in MVA-BN-RSV-miRb39/41. Expression of N/M2-1 is downregulated particularly well as expected since its expression is driven by an early promoter.

6.3 Yields of Modified MVA-BN-RSV Recombinants Yields of recombinant MVA from CEF cells infected with MVA-BN-RSV, MVA-BN-RSV-miRb1/2 and MVA-BN-RSV-miRb39/41 were determined at two different MOIs, i.e. 0.1 and 0.01, on day 3 and 4 p.i.

As shown in FIG. 17, MVA-BN-RSV produced significantly reduced yields compared to wildtype MVA-BN both at an MOI of 0.1 and 0.01 on day 3 and 4 p.i. (decrease about 4.2-fold to 21.5-fold, see the table in FIG. 17, “MVA-BN vs. -RSV”). Compared to MVA-BN-RSV, however, the yield of MVA-BN-RSV-miRb1/2 was increased by about 1.2-fold to 4.2-fold (“MVA-RSV-miRb1/2 vs. MVA-RSV”). The yield of MVA-BN-RSV-miRb39/41 further increased over MVA-BN-RSV-miRb1/2 (at least at MOI 0.1 on day 3 and 4 p.i., and at MOI 0.01 on day 4 p.i.) between 1.2-fold and 2.6-fold (“MVA-RSV-miRb39/41 vs. -miRb1/2”). Best increases over MVA-BN-RSV were observed with MVA-BN-RSV-miRb39/41 at a MOI of 0.1 on day 3 and 4 p.i. (about 3.2-fold and 6.8-fold, respectively) (“MVA-RSV-miRb39/41 vs. MVA-RSV”).

Nevertheless, MVA-BN-RSV-miRb39/41 did not completely regain the replication behavior of MVA-BN (see the Table in FIG. 17, “MVA-BN vs. RSV-miRb39/41): The yield of MVA-BN-RSV-miRb39/41 was still reduced compared to MVA-BN by about 3.2-fold (MOI 0.1) and 2.6-fold (MOI 0.01).

In conclusion, the best effect produced by miRNA target sequences with regard to production yields was obtained with MVA-BN-RSV-miRb39/41 at a MOI of 0.1 and on day 4 p.i.

6.4 Immunogenicity of RSV Proteins from Modified MVA-BN-RSV

### 6.4.1 T Cell Assay

In order to examine immunogenicity of the RSV transgene products from MVA-BN-RSV-miRb39/41 and MVA-BN-RSV-miRb1/2 we conducted a mouse immunization experiment.

When produced in the BALB/c strain of mice the M2-1 protein harbors a strong, immunodominant CD8 T cell epitope. Thus, analysis of this epitope provided a sensitive assay with a wide dynamic range for determining the RSV M2-1-specific CD8 T cell response.

CD8 T cell responses in BALB/mice specific for RSV M2-1 or MVA E3 (used as a vector control) were analyzed by Dextramer staining. Mouse PBMCs were collected and stained on day 7 post prime as well as on day 7 and 13 after a boost on day 21 (i.e., day 28 and day 34 after the first immunization).

As shown in FIG. 18 (left panels), MVA-BN-RSV, MVA-BN-RSV-miRb1/2 and MVA-BN-RSV-miRb39/41 induced similar frequencies of CD8⁺ T cells specific for RSV M2-1 at all times analyzed. Thus, immunogenicity of RSV N/M2-1 was not affected in vivo by miRNA target sequences present in the modified MVA-BN-RSV recombinants.

Furthermore, MVA-BN-RSV, MVA-BN-RSV-miRb1/2 and MVA-BN-RSV-miRb39/41 induced similar frequencies of E3-specific CD8⁺ T cells (FIG. 18 right panels), demonstrating that the T cell response to the MVA vector backbone was comparable and that immunizations with the different recombinants were in general similarly effective. Additionally, analyses of frequencies of memory T cell phenotype subsets based on the expression pattern of certain cell surface markers (CD4, CD8, CD44, CD62L, CD127, CD29, CX3CR1) did not reveal differences between the three MVA-BN-RSV recombinants.

### 6.4.2 Intracellular Cytokine Staining and ELISpot Analysis

To additionally assess functional responses of CD8⁺ T cells in terms of cytokine production and to quantitatively compare the CD8 T cell responses against RSV F and G proteins, mice splenocytes were collected on day 13 post boost and immediately stimulated with peptides derived from RSV G, F or M2-1, or from MVA E3 (vector control). Responses were analyzed by intracellular cytokine staining (ICCS) and ELISpot analysis.

In mice immunized with MVA-BN-RSV, MVA-BN-RSV-miRb1/2 or MVA-BN-RSV-miRb39/41, the overall frequencies of CD44⁺ IFN-γ⁺ CD8⁺ T cells specific for each of the RSV G, F and N/M2-1 proteins or MVA E3 were very similar between the groups of mice immunized with one of the three MVA-BN-RSV recombinants (FIG. 19A). After stimulation with RSV F-derived peptides, the overall frequencies of IFN-γ⁺ CD8⁺ T cells were the lowest among the antigens tested (<2%) while those obtained with RSV N/M2-1-derived peptides were highest (FIG. 19A). Besides, the patterns of surface markers CD62L, CD127 and CX3CR1 were very similar between all groups of mice.

T cell responses in the immunized mice were also analyzed using the ELISPOT assay, encompassing analysis of CD4 and CD8 T cells. The results are shown in FIG. 19B. ELISpot analyses after stimulation of splenocytes with the RSV M2-1 derived peptide resulted in IFN-γ⁺ spot-forming colonies too numerous to count (and therefore not shown in FIG. 19B). This result confirmed that the RSV N/M2-1 protein induced strong CD8 T cell responses which was in agreement with reports by others that RSV M2-1 contains the immunodominant epitope in H-2d mice (36, 37).

ELISpot analysis of splenocytes after stimulation with peptides derived from RSV G and F as well as MVA E3 confirmed that responses to RSV G peptides were stronger than those to RSV F peptides (FIG. 19B). As with ICCS (FIG. 19A and discussed above), no differences in the frequencies of T cells specific for each of the RSV G and F proteins and MVA E3 between the groups of mice immunized with one of the three MVA-BN-RSV recombinants were observed (FIG. 19B).

### 6.4.3 ELISA and PRNT Analysis

Humoral responses against the encoded RSV proteins were analyzed by an ELISA for immunoglobulin G (IgG) antibody binding to whole RSV as antigen and by determining the titer of antibodies neutralizing infectious RSV in vitro. RSV- and MVA-specific IgG titers were analyzed one day before prime (day −1), day 20 post prime, and day 34 post prime (i.e., day 13 post boost).

After prime or boost, no differences in total RSV-specific IgG titers were observed in sera of mice immunized with one of the three MVA-BN-RSV recombinants (FIG. 20A left). A similar result was obtained for MVA-specific IgG titers (FIG. 20A right).

RSV-specific neutralizing antibody titers were determined in sera of immunized mice at day 34 post prime (i.e., day 13 post boost) by a plaque reduction neutralization test (PRNT).

No differences in the amounts of neutralizing antibodies induced after immunization of mice with one of the three different MVA-BN-RSV recombinants were detectable (FIG. 20B). The rate of seroconversion with respect to RSV-neutralizing antibodies was 100% in all groups of immunized mice (see the Table in FIG. 20B).

6.4.4 Conclusion from Immunogenicity Studies

In summary, T cell responses, including the frequencies and functionality of RSV-specific CD8⁺ T cells, and the induction of total RSV-specific IgG as well as neutralizing antibodies were highly comparable between groups of mice immunized with one of the three different MVA-BN-RSV recombinants. Thus, the addition of miRNA target sequences within the 3′-UTR of transgenes did not negatively affect the immunogenicity of the respective transgene products in mice compared to that of transgene products from in the non-modified MVA-BN-RSV construct. The results altogether showed that downregulation of cytotoxic transgene expression mediated by miRNA target sequences positively affected MVA yields from CEF cells without detectably affecting the immunogenicity of the respective transgene products in vivo.

Final remark: Several documents are cited throughout the text of this specification. Each of the documents cited herein (including all patents, patent applications, scientific publications, manufacturer's specifications, instructions, etc.) are hereby incorporated by reference in their entirety. To the extent, the material incorporated by reference contradicts or is inconsistent with this specification, the specification will supersede any such material. Nothing herein is to be construed as an admission that the invention is not entitled to antedate such disclosure by virtue of prior invention.

### REFERENCES

1. Cottingham M G, Carroll F, Morris S J, Turner A V, Vaughan A M, Kapulu M C, Colloca S, Siani L, Gilbert S C, Hill A V. 2012. Preventing spontaneous genetic rearrangements in the transgene cassettes of adenovirus vectors. Biotechnol Bioeng 109:719-28.
2. Wyatt L S, Belyakov I M, Earl P L, Berzofsky J A, Moss B. 2008. Enhanced cell surface expression, immunogenicity and genetic stability resulting from a spontaneous truncation of HIV Env expressed by a recombinant MVA. Virology 372:260-272.
3. Meisinger-Henschel C, Schmidt M, Lukassen S, Linke B, Krause L, Konietzny S, Goesmann A, Howley P, Chaplin P, Suter M, Hausmann J. 2007. Genomic sequence of chorioallantois vaccinia virus Ankara, the ancestor of modified vaccinia virus Ankara. J Gen Virol 88:3249-3259.
4. Suter M, Meisinger-Henschel C, Tzatzaris M, HOlsemann V, Lukassen S, Wulff N H, Hausmann J, Howley P, Chaplin P. 2009. Modified vaccinia Ankara strains with identical coding sequences actually represent complex mixtures of viruses that determine the biological properties of each strain. Vaccine 27:7442-7450.
5. Pollard A J, Launay 0, Lelievre J D, Lacabaratz C, Grande S, Goldstein N, Robinson C, Gaddah A, Bockstal V, Wiedemann A, Leyssen M, Luhn K, Richert L, Betard C, Gibani M M, Clutterbuck E A, Snape M D, Levy Y, Douoguih M, Thiebaut R, group EEs. 2021. Safety and immunogenicity of a two-dose heterologous Ad26.ZEBOV and MVA-BN-Filo Ebola vaccine regimen in adults in Europe (EBOVAC2): a randomised, observer-blind, participant-blind, placebo-controlled, phase 2 trial. Lancet Infect Dis 21:493-506.
6. Baek D, Villen J, Shin C, Camargo F D, Gygi S P, Bartel D P. 2008. The impact of microRNAs on protein output. Nature 455:64-71.
7. Selbach M, Schwanhausser B, Thierfelder N, Fang Z, Khanin R, Rajewsky N. 2008. Widespread changes in protein synthesis induced by microRNAs. Nature 455:58-63.
8. Geisler A, Fechner H. 2016. MicroRNA-regulated viral vectors for gene therapy. World J Exp Med 6:37-54.
9. Barnes D, Kunitomi M, Vignuzzi M, Saksela K, Andino R. 2008. Harnessing endogenous miRNAs to control virus tissue tropism as a strategy for developing attenuated virus vaccines. Cell Host Microbe 4:239-248.
10. Edge R E, Falls T J, Brown C W, Lichty B D, Atkins H, Bell J C. 2008. A let-7 MicroRNA-sensitive vesicular stomatitis virus demonstrates tumor-specific replication. Mol Ther 16:1437-1443.
11. Kelly E J, Hadac E M, Greiner S, Russell S J. 2008. Engineering microRNA responsiveness to decrease virus pathogenicity. Nat Med 14:1278-1283.
12. Langlois R A, Varble A, Chua M A, Garcia-Sastre A, tenOever B R. 2012. Hematopoietic-specific targeting of influenza A virus reveals replication requirements for induction of antiviral immune responses. Proc Natl Acad Sci USA 109:12117-12122.
13. Perez J T, Pham A M, Lorini M H, Chua M A, Steel J, tenOever B R. 2009. MicroRNA-mediated species-specific attenuation of influenza A virus. Nat Biotechnol 27:572-576.
14. Reid C A, Boye S L, Hauswirth W W, Lipinski D M. 2017. miRNA-mediated post-transcriptional silencing of transgenes leads to increased adeno-associated viral vector yield and targeting specificity. Gene Ther 24:462-469.
15. Guimaro M C, Afione S A, Tanaka T, Chiorini J A. 2020. Rescue of Adeno-Associated Virus Production by shRNA Cotransfection. Hum Gene Ther 31:1068-1073.
16. Backes S, Shapiro J S, Sabin L R, Pham A M, Reyes I, Moss B, Cherry S, tenOever B R. 2012. Degradation of host microRNAs by poxvirus poly(A) polymerase reveals terminal RNA methylation as a protective antiviral mechanism. Cell Host Microbe 12:200-210.
17. Grinberg M, Gilad S, Meiri E, Levy A, Isakov O, Ronen R, Shomron N, Bentwich Z, Shemer-Avni Y. 2012. Vaccinia virus infection suppresses the cell microRNA machinery. Arch Virol 157:1719-1727.
18. Chen J S, Li H C, Lin S I, Yang C H, Chien W Y, Syu C L, Lo S Y. 2015. Cleavage of Dicer protein by 17 protease during vaccinia virus infection. PLoS ONE 10:e0120390.
19. Futami M, Sato K, Miyazaki K, Suzuki K, Nakamura T, Tojo A. 2017. Efficacy and Safety of Doubly-Regulated Vaccinia Virus in a Mouse Xenograft Model of Multiple Myeloma. Mol Ther Oncolytics 6:57-68.
20. Hikichi M, Kidokoro M, Haraguchi T, Iba H, Shida H, Tahara H, Nakamura T. 2011. MicroRNA regulation of glycoprotein B5R in oncolytic vaccinia virus reduces viral pathogenicity without impairing its antitumor efficacy. Mol Ther 19:1107-1115.
21. Assarsson E, Greenbaum J A, Sundstrom M, Schaffer L, Hammond J A, Pasquetto V, Oseroff C, Hendrickson R C, Lefkowitz E J, Tscharke D C, Sidney J, Grey H M, Head S R, Peters B, Sette A. 2008. Kinetic analysis of a complete poxvirus transcriptome reveals an immediate-early class of genes. Proc Natl Acad Sci USA 105:2140-2145.
22. Engelstad M, Smith G L. 1993. The vaccinia virus 42-kDa envelope protein is required for the envelopment and egress of extracellular virus and for virus virulence. Virology 194:627-637.
23. Chakrabarti S, Sisler J R, Moss B. 1997. Compact, synthetic, vaccinia virus early/late promoter for protein expression. Biotechniques 23:1094-1097.
24. Wennier S T, Brinkmann K, SteinhsuBer C, Maylsnder N, Mnich C, Wielert U, Dirmeier U, Hausmann J, Chaplin P, Steigerwald R. 2013. A novel naturally occurring tandem promoter in modified vaccinia virus Ankara drives very early gene expression and potent immune responses. PLoS ONE 8:e73511.
25. Burnside J, Ouyang M, Anderson A, Bernberg E, Lu C, Meyers B C, Green P J, Markis M, Isaacs G, Huang E, Morgan R W. 2008. Deep sequencing of chicken microRNAs. BMC Genomics 9:185.
26. Glazov E A, Cottee P A, Barris W C, Moore R J, Dalrymple B P, Tizard M L. 2008. A microRNA catalog of the developing chicken embryo identified by a deep sequencing approach. Genome Res 18:957-964.
27. Hicks J A, Tembhurne P, Liu H C. 2008. MicroRNA expression in chicken embryos. Poult Sci 87:2335-2343.
28. Lim W, Song G. 2014. Identification of novel regulatory genes in development of the avian reproductive tracts. PLoS ONE 9:e96175.
29. Meunier J, Lemoine F, Soumillon M, Liechti A, Weier M, Guschanski K, Hu H, Khaitovich P, Kaessmann H. 2013. Birth and expression evolution of mammalian microRNA genes. Genome Res 23:34-45.
30. Peng X, Gao Q S, Zhou L, Chen Z H, Lu S, Huang H J, Zhan C Y, Xiang M. 2015. MicroRNAs in avian influenza virus H9N2-infected and non-infected chicken embryo fibroblasts. Genet Mol Res 14:9081-9091.
31. Wu N, Zhu Q, Chen B, Gao J, Xu Z, Li D. 2017. High-throughput sequencing of pituitary and hypothalamic microRNA transcriptome associated with high rate of egg production. BMC Genomics 18:255.
32. Baur K, Brinkmann K, Schweneker M, Pstzold J, Meisinger-Henschel C, Hermann J, Steigerwald R, Chaplin P, Suter M, Hausmann J. 2010. Immediate-early expression of a recombinant antigen by modified vaccinia virus ankara breaks the immunodominance of strong vector-specific B8R antigen in acute and memory CD8 T-cell responses. J Virol 84:8743-8752.
33. Jessen B, Faller S, Krempl C D, Ehl S. 2011. Major histocompatibility complex-dependent cytotoxic T lymphocyte repertoire and functional avidity contribute to strain-specific disease susceptibility after murine respiratory syncytial virus infection. J Virol 85:10135-43.
34. Openshaw P J, Anderson K, Wertz G W, Askonas B A. 1990. The 22,000-kilodalton protein of respiratory syncytial virus is a major target for Kd-restricted cytotoxic T lymphocytes from mice primed by infection. J Virol 64:1683-9.
35. Meisinger-Henschel C, Spsth M, Lukassen S, Wolferststter M, Kachelriess H, Baur K, Dirmeier U, Wagner M, Chaplin P, Suter M, Hausmann J. 2010. Introduction of the six major genomic deletions of modified vaccinia virus Ankara (MVA) into the parental vaccinia virus is not sufficient to reproduce an MVA-like phenotype in cell culture and in mice. J Virol 84:9907-9919.
36. O'Connor M, Peifer M, Bender W. 1989. Construction of large DNA segments in Escherichia coli. Science 244:1307-1312.
37. Wolferststter M, Schweneker M, Spsth M, Lukassen S, Klingenberg M, Brinkmann K, Wielert U, Lauterbach H, Hochrein H, Chaplin P, Suter M, Hausmann J. 2014. Recombinant modified vaccinia virus ankara generating excess early double-stranded RNA transiently activates protein kinase R and triggers enhanced innate immune responses. J Virol 88:14396-14411.

We claim:

1. A recombinant Modified Vaccinia Virus Ankara (MVA) comprising a nucleotide sequence comprising a transgene operably linked to a poxvirus promoter, the nucleotide sequence further comprising a series of miRNA target sequences arranged in a heterologous miRblock that is linked to the transgene, wherein each miRNA target sequence corresponds to a miRNA in a eukaryotic MVA producer cell, wherein at least one of the miRNA target sequences in the miRblock is capable of mediating downregulation of the transgene's expression in the eukaryotic MVA producer cell.

2. A transcriptional unit comprising a nucleotide sequence comprising a transgene operably linked to a poxvirus promoter, the nucleotide sequence further comprising a series of miRNA target sequences arranged in a heterologous miRblock that is linked to the transgene, wherein each miRNA target sequence corresponds to a miRNA sequence in a eukaryotic MVA producer cell, wherein at least one of the miRNA target sequences in the miRblock is capable of mediating downregulation of the transgene's expression in the eukaryotic MVA producer cell.

3. A series of miRNA target sequences arranged in a heterologous miRblock, wherein each miRNA target sequence corresponds to a miRNA in a eukaryotic MVA producer cell, wherein at least one of the miRNA target sequences in the miRblock is capable of mediating downregulation of the expression of a transgene linked to the miRblock in the eukaryotic MVA producer cell.

4. The recombinant MVA of claim 1, wherein at least one miRNA target sequence corresponds to the sequence of the miRNA at a nucleotide sequence similarity of from about 80 to 100%, preferably of from about 90 to 100%, more preferably of from about 95 to 100%, most preferably of about 100%.

5. The recombinant MVA of claim 4, wherein at least one miRNA target sequence is selected from the group consisting of nucleotide sequences as depicted in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9.

6. The recombinant MVA of claim 4, wherein the miRblock comprises or consists of a nucleotide sequence selected from the group consisting of nucleotide sequences as depicted in SEQ ID NO: 48, SEQ ID NO: 49, SEQ ID NO: 51, SEQ ID NO: 52, SEQ ID NO: 53, SEQ ID NO: 54, and SEQ ID NO: 55.

7. The recombinant MVA of claim 1, wherein the transgene encodes a protein derived from respiratory syncytial virus (RSV), or an antigenic part thereof, preferably selected from the group consisting of RSV G(A), G(B), F, N, and M2-1 protein, and a N/M2-1 fusion protein.

8. The recombinant MVA of claim 1, wherein the eukaryotic MVA producer cell is a primary avian cell, preferably a chicken embryo fibroblast (CEF) cell, or a permanent avian cell line, preferably a DF-1 or a quail cell.

9. The recombinant MVA of claim 1, wherein the promoter is an immediate-early promoter selected from the group consisting of Pr13.5long, Pr1328, PrLE1 (pHyb) promoters, preferably is a Pr13.5long promoter.

10. A process for producing a recombinant MVA of claim 1, comprising the steps of:
(1) providing a series of miRNA target sequences arranged in a miRblock;
(2) preparing a transcriptional unit using the miRblock provided in step (1);
(3) inserting the transcriptional unit prepared in step (2) into an MVA;
(4) infecting a eukaryotic MVA producer cell with the MVA obtained in step (3) and propagating the same; and
(5) harvesting the recombinant MVA propagated in step (4).

11. A method for downregulating the expression of an MVA encoded transgene in a eukaryotic MVA producer cell in vitro, comprising infecting said cell with the recombinant MVA of claim 1, wherein the miRNA target sequence is selected from the group consisting of nucleotide sequences as depicted in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9.

12. A method for downregulating the expression of an MVA encoded transgene in a eukaryotic MVA producer cell in vitro, comprising infecting said cell with the recombinant MVA of claim 1, wherein the miRblock comprises or consists of a nucleotide sequence selected from the group consisting of nucleotide sequences as depicted in SEQ ID NO: 48, SEQ ID NO: 49, SEQ ID NO: 51, SEQ ID NO: 52, SEQ ID NO: 53, SEQ ID NO: 54, and SEQ ID NO: 55.

13. A pharmaceutical composition or a vaccine comprising the recombinant MVA of claim 1.

14. (canceled)

15. A method of treating or preventing an infectious disease or cancer in a subject, comprising administering to said subject a recombinant MVA of claim 1.

16. (canceled)
